(19) United States
(12) Patent Application Publication
(10) Pub. No.: US 2026/0134175 A1
(43) Pub. Date: May 14, 2026

(54) GENERATING TIME-TO-EVENT DETERMINATIONS BASED ON PROFILES MAINTAINED IN A SECURED NETWORK LOCATION

(71) Applicant: AML JV, LLC, Aurora, CO (US)

(72) Inventors: Justin DALE, Aurora, CO (US); Jamie REUBEN, Aurora, CO (US); Md Nazmul ISLAM, Aurora, CO (US)

(73) Assignee: AML JV, LLC, Aurora, CO (US)

(21) Appl. No.: 19/383,157

(22) Filed: Nov. 7, 2025

Related U.S. Application Data
(60) Provisional application No. 63/718,477, filed on Nov. 8, 2024.
(60) Provisional application No. 63/724,124, filed on Nov. 22, 2024.

(51) Int. Cl. G06F 30/27; G16H 50/70

(52) U.S. Cl. CPC G06F 30/27; G16H 50/70

(57) ABSTRACT

Disclosed are systems for predicting indicators indicative of a condition (e.g., a disease, etc.). In some examples, a system can be configured to obtain a first set of patient data associated with a plurality of patients, generate a set of training data by determining, from the first set of patient data, one or more features from among a plurality of features that are correlated with expression of a target protein. The system can then be configured to train a plurality of models and evaluate performance of each model relative to the other models. A model can then be selected based on its performance.

### CROSS-REFERENCE TO RELATED APPLICATIONS

The present disclosure claims the benefit of, and priority to, U.S. Provisional Patent Application No. 63/718,477, titled “SYSTEMS AND METHODS FOR MODELING RESPONSES BASED ON ADMINISTRATION OF AN AGENT,” filed Nov. 8, 2024, and U.S. Provisional Patent Application No. 63/724,124, titled “SYSTEMS AND METHODS FOR MODELING RESPONSES BASED ON ADMINISTRATION OF AN AGENT,” filed Nov. 22, 2024, all of which are hereby incorporated by reference in their entirety and for all purposes.

### TECHNICAL FIELD

This application relates generally to systems and methods for predicting indicators such as biomarkers that are correlated with a disease and, in some implementations, to techniques for predicting indicators in response to analyzing multiple treatment profiles.

### BACKGROUND

Early detection and treatment of diseases such as cancers (including acute myelogenous leukemia (AML), among others) is generally regarded as being one of the most important factors in successfully treating a patient and improve their health outcomes (e.g., through lifespan extension and, in some cases, achieving disease remission). However, it can be difficult to identify diseases at these early stages, particularly those where patients are not experiencing classic symptoms (e.g., pain, fatigue, etc.). For example, because of the high dimensionality of biological data, which often includes numerous potential biomarkers that may not have a straightforward relationship with each other, it can be difficult to determine whether an individual has a disease or if there are other factors that are contributing to their state. This complexity is compounded by patient-specific variations, such as genetic differences and environmental influences, which can lead to inconsistent biomarker measurements across individuals. Furthermore, the presence of confounding factors in clinical data, such as comorbidities or treatment histories, can obscure the true association between a biomarker and a disease.

Also, diseases such as AML are treated in coordination with treatment plans developed by clinicians and based on standard of care therapies. These treatment plans can include the use of therapies such as administration of agents targeting specific molecules or pathways involved in the growth and survival of the disease, stem cell transplants, and/or the like. Typically, these treatments are administered in conjunction with monitoring of the response of the patient through diagnostic testing and updated to optimize a patient's outcome. But it is often difficult for clinicians to determine which therapy will be most effective in treating the targeted disease. This can lead to therapies being applied to patients that are less efficient and that do not result in an optimal outcome.

### SUMMARY

For the aforementioned reasons, there is a need for systems and methods that can predict indicators for a disease in response to analyzing multiple treatment profiles. The present disclosure addresses these difficulties by identifying features (sometimes referred to as a “variable selection problem”) that are correlated with expression of one or more target proteins and testing multiple models (e.g., machine learning models, etc.) to identify and select one model that outperforms the others at predicting whether a specific individual has a disease. And once deployed, the selected model provides multiple benefits across various technical fields, particularly in medicine and diagnostic analysis of patients. First, diagnostic accuracy can be improved by allowing for the detection of diseases at earlier stages through patterns or combinations of indicator levels that might not be significant individually and/or might not be identifiable through traditional statistical analysis (referred to as latent patterns). Second, it facilitates the development of targeted therapies by revealing which indicators are linked to specific disease pathways, allowing for the creation or targeted testing of drugs that address these precise biological indicators. Third, understanding indicator correlations can uncover new disease mechanisms or subtypes, leading to improvements in how diseases are classified and treated.

In some aspects, described herein is a system for selecting an optimized system configuration based on performance metrics associated with time-to-event determinations generated by a plurality of systems using treatment profiles. The system can include one or more processors. The one or more processors can be configured to determine a set of features from among a plurality of features represented across a plurality of treatment profiles. The set of features can be correlated with time-to-event determinations (e.g., a point at which a corresponding individual is expected to survive, a point at which one or more effects from a condition or treatment will be experienced, etc.) that satisfy a correlation threshold. In examples, the one or more processors can generate an analysis dataset including a plurality of second treatment profiles based on the set of features that are correlated with the time-to-event determinations. In some examples, the one or more processors can be configured to train a plurality of models based on the analysis dataset to output the time-to-event determinations. The one or more processors can be configured to determine, for each model of the plurality of models configured with the analysis dataset or a different dataset, a plurality of performance metrics based on the time-to-event determinations. In examples, the one or more processors can be configured to configure the system to receive different treatment profiles to determine different time-to-event determinations by: selecting an optimized model from among the plurality of models based on the plurality of performance metrics; and for at least one treatment profile, generating an indication of a therapy response based on the optimized model and a time-to-event determination generated by the optimized model. The time-to-event determination can represent a counterfactual analysis of the treatment profile in a first case where a therapy is administered and in a second case where a therapy is not administered

In at least some aspects, the set of features can represent individuals (e.g., a state of the individuals) having a condition. The one or more processors can be further configured to filter the plurality of features based on one or more criteria indicating administration of an agent that is configured to adjust progression of the condition. The one or more processors can be configured to determine the set of features in response to filtering the plurality of features.

In some aspects, the one or more processors configured to filter the plurality of features can be configured to filter the plurality of features based on a plurality of criteria indicating administration of a plurality of agents that are configured to adjust progression of the condition when administered in combination.

In at least some aspects, the one or more processors configured to generate the analysis dataset can be configured to generate a plurality of additional features for one or more of the plurality of second treatment profiles. Each additional feature can include one or more synthetic features that augment the features for the respective one or more of the plurality of second treatment profiles.

In some aspects, the one or more processors configured to train the plurality of models can be configure to, for each model: determine a combination of features and additional features that satisfy each model of the plurality of models; and train the plurality of models using the combination of features and additional features.

In at least some aspects, each performance metric of the plurality of performance metrics can include a prediction of survival at a point in time after diagnosis of a condition. The one or more processors configured to determine the plurality of performance metrics for each profile can be configured to determine an average performance metric corresponding to each profile to determine a treatment response classification. The one or more processors configured to determine the indication of the therapy response can be configured to determine, for each profile, an administration response based on the average performance metric corresponding to each profile.

In at least some aspects, the one or more processors configured to determine the administration response for each profile can be configured to classify the administration response for each profile as being favorable, intermediate, or not favorable based on the average performance metric for each profile.

In aspects, a method for selecting an optimized system configuration based on performance metrics associated with time-to-event determinations generated by a plurality of systems using treatment profiles is described. The method can include determining, by one or more processors, a set of features from among a plurality of features represented across a plurality of treatment profiles. The set of features can be correlated with time-to-event determinations that satisfy a correlation threshold. In examples, the method can include generating, by the one or more processors, an analysis dataset including a plurality of second treatment profiles based on the set of features that are correlated with the time-to-event determinations. In some examples, the method can include training, by the one or more processors, a plurality of models based on the analysis dataset to output the time-to-event determinations. In examples, the method can include determining, by the one or more processors, for each model of the plurality of models configured with the analysis dataset or a different dataset, a plurality of performance metrics based on the time-to-event determinations. In some examples, the method can include configuring, by the one or more processors, the system to receive different treatment profiles to determine different time-to-event determinations by: selecting, by the one or more processors, an optimized model from among the plurality of models based on the plurality of performance metrics; and for at least one treatment profile, generating, by the one or more processors, an indication of a therapy response based on the optimized model and a time-to-event determination generated by the optimized model.

In some aspects, the set of features can represent individuals having a condition. The method can further include filtering, by the one or more processors, the plurality of features based on one or more criteria indicating administration of an agent that is configured to adjust progression of the condition. In examples, the method can include determining, by the one or more processors, the set of features in response to filtering the plurality of features.

In aspects, filtering the plurality of features can include filtering, by the one or more processors, the plurality of features based on a plurality of criteria indicating administration of a plurality of agents that are configured to adjust progression of the condition when administered in combination.

In at least some aspects, generating the analysis dataset can include generating, by the one or more processors, a plurality of additional features for one or more of the plurality of second treatment profiles. Each additional feature can include one or more synthetic features that augment the features for the respective one or more of the plurality of second treatment profiles.

In some aspects, training the plurality of models can include, for each model: determining, by the one or more processors, a combination of features and additional features that satisfy each model of the plurality of models; and training, by the one or more processors, the plurality of models using the combination of features and additional features.

In aspects, each performance metric of the plurality of performance metrics can include a prediction of survival at a point in time after diagnosis of a condition. Determining the plurality of performance metrics for each profile can include determining, by the one or more processors, an average performance metric corresponding to each profile to determine a treatment response classification. Determining the indication of the therapy response can include determining, by the one or more processors, for each profile, an administration response based on the average performance metric corresponding to each profile.

In at least some aspects, determining the administration response for each profile can include classifying, by the one or more processors, the administration response for each profile as being favorable, intermediate, or not favorable based on the average performance metric for each profile.

In some aspects, one or more non-transitory computer-readable mediums are storing instructions thereon are disclosed. The instructions can, when executed by one or more processors, cause the one or more processors to perform operations. The operations can include determining a set of features from among a plurality of features represented across a plurality of treatment profiles. The set of features can be correlated with time-to-event determinations that satisfy a correlation threshold. In examples, the operations can include generating an analysis dataset including a plurality of second treatment profiles based on the set of features that are correlated with the time-to-event determinations. In at least some examples, the operations can include training a plurality of models based on the analysis dataset to output the time-to-event determinations. In examples, the operations can include determining for each model of the plurality of models configured with the analysis dataset or a different dataset, a plurality of performance metrics based on the time-to-event determinations. In at least some examples, the operations can include configuring the system to receive different treatment profiles to determine different time-to-event determinations by: selecting an optimized model from among the plurality of models based on the plurality of performance metrics; and for at least one treatment profile, generating an indication of a therapy response based on the optimized model and a time-to-event determination generated by the optimized model.

In some aspects, the set of features represent individuals having a condition. The instructions can further cause the one or more processors to: filter the plurality of features based on one or more criteria indicating administration of an agent that is configured to adjust progression of the condition. The instructions can cause the one or more processors to determine the set of features in response to filtering the plurality of features.

In aspects, the instructions that cause the one or more processors to filter the plurality of features can cause the one or more processors to filter the plurality of features based on a plurality of criteria indicating administration of a plurality of agents that are configured to adjust progression of the condition when administered in combination.

In at least some aspects, the instructions that cause the one or more processors to generate the analysis dataset can cause the one or more processors to generate a plurality of additional features for one or more of the plurality of second treatment profiles, each additional feature including one or more synthetic features that augment the features for the respective one or more of the plurality of second treatment profiles.

In aspects, the instructions that cause the one or more processors to train the plurality of models can cause the one or more processors to, for each model: determine a combination of features and additional features that satisfy each model of the plurality of models; and train the plurality of models using the combination of features and additional features.

In some aspects, each performance metric of the plurality of performance metrics can include a prediction of survival at a point in time after diagnosis of a condition. The instructions that cause the one or more processors to determine the plurality of performance metrics for each profile can cause the one or more processors to determine an average performance metric corresponding to each profile to determine a treatment response classification. In examples, the instructions that cause the one or more processors to determine the indication of the therapy response can cause the one or more processors to determine, for each profile, an administration response based on the average performance metric corresponding to each profile.

By virtue of the implementation of the techniques described herein, systems can be configured to more accurately identify individuals as having (or not having) a disease (e.g., AML and/or other diseases) based on measurements of a variety of indicators. For example, through the correlation of indicators with a specific protein indicative of a disease, the precision of disease diagnosis can be improved over conventional techniques by providing a clearer picture of disease pathogenesis through the identification of multiple related indicators, allowing for earlier detection and intervention. And the implementation of certain techniques described herein can allow for the identification of diseases represented by latent patterns within high-dimensional datasets by efficiently managing complexity, capturing non-linear relationships, and adapting to new data that would otherwise be unobservable through conventional statistical analysis. Further, the presently-disclosed techniques facilitate the development of more accurate prognostic models, where the combination of indicators can predict disease progression or individual outcomes with greater confidence than a single indicator. And by using trained models as describe herein, computational resources that would be needed to sequence samples of the individual to measure the expression of a target protein associated with a disease can be conserved as combinations of simpler, less computationally-intensive indicator measurements can be used in place of these sequenced samples.

Embodiments described herein include systems and methods for modeling responses based on administration of an agent and can provide any number of additional or alternative benefits as well. When implemented, these systems and methods can address inefficiencies involved in conventional treatment planning. For example, the system sand methods described herein can allow for the determination of whether an agent will result in a favorable (e.g., life extending) or not favorable response based on one or more indicators (e.g., biomarkers) measured in the patient.

In some aspects, a system is described for selecting an optimized system architecture when determining whether indicators are represented in protected treatment profiles based on performance of a plurality of systems having different system configurations. The system can include one or more processors. The one or more processors can be configured to obtain a first set of data associated with a plurality of profiles. Each profile can include a plurality of indicators for a plurality of indicator types. The one or more processors can be configured to generate a set of training data by determining one or more indicator types for one or more indicators that are correlated with expression of target proteins. In examples, the one or more processors can be configured to train a first system having a first architecture with a first subset of the set of training data and a second system having a second architecture that is at least in part different from the first architecture with a second subset of the set of training data based on the set of training data to generate, from the first system and the second system, the outputs indicating that representing profiles indicate expression of the target proteins based on indicators that are not represented in the set of training data. In some examples, in response to comparing a first degree of precision of the outputs of the first system to a second degree of precision of the outputs of the second system to determine that the first system or the second system includes an optimized system that has a higher degree of precision for generating the outputs indicating the expression of the target proteins the one or more processors can be configured to cause the system to: select the optimized system from among the plurality of systems based on input of data associated with a different profile in response to comparing the first degree of precision and the second degree of precision; and for at least one different treatment profile, generate an indication of a therapy response based on the optimized system and an output generated by the optimized system.

In some aspects, the techniques described herein relate to a system, wherein the one or more processors configured to generate the set of training data are configured to: execute a supervised feature selection algorithm or an unsupervised feature selection algorithm in accordance with the first set of data to identify the one or more indicator types correlated with expression of the target proteins; and select the one or more indicator types in response to executing the supervised feature selection algorithm or the unsupervised feature selection algorithm.

In aspects, the techniques described herein relate to a system, wherein the one or more processors configured to generate the set of training data are configured to: calculate a feature importance estimate for each indicator of the plurality of indicator types; and select the one or more indicator types based on the feature importance estimate for each indicator of the plurality of indicator types.

In at least some aspects, the techniques described herein relate to a system, the one or more processors configured to select the one or more indicator types are configured to: compare the feature importance estimate for each indicator of the plurality of indicator types to a threshold value to determine that the one or more indicator types satisfy the threshold value; and select the one or more indicator types based on the feature importance estimate satisfying the threshold value.

In some aspects, the techniques described herein relate to a system, wherein the one or more processors configured to train the first system and the second system are configured to: train the first system, where the first system includes a first tree-based model that is trained in accordance with gradient boosting techniques; and train the second system, where the second system including a logistic regression model or a penalized logistic regression model.

In aspects, the techniques described herein relate to a system, wherein the first system includes an ensemble of models, and wherein the one or more processors configured to train the first system or the second system are configured to: train the ensemble of models to generate the outputs indicative of probabilities that individuals express a target protein correlated with a condition.

In at least some aspects, the techniques described herein relate to a system, wherein the one or more indicator types include one or more first indicator types, wherein the one or more processors configured to generate the set of training data are configured to: calculate an indicator importance estimate for each indicator of the plurality of indicator types; and select the one or more first indicator types based on the indicator importance estimate for each indicator of the plurality of indicator types, wherein, in response to comparing the first degree of precision of the outputs of the first system and the second degree of precision of the outputs of the second system, the one or more processors are configured to: determine that the first system outperforms the second system based on a comparison of the first degree of precision with the second degree of precision; and update the first system based on the one or more first indicator types and one or more second indicator types from among the plurality of indicator types.

In some aspects, the techniques described herein relate to a method for selecting an optimized system architecture when determining whether indicators are represented in protected treatment profiles based on performance of a plurality of systems having different system configurations. The method can include obtaining, by one or more processors, a first set of data associated with a plurality of profiles, each profile including a plurality of indicators for a plurality of indicator types. In examples, the method can include generating, by the one or more processors, a set of training data by determining one or more indicator types for one or more indicators that are correlated with expression of target proteins. In some examples, the method can include training, by the one or more processors, a first system having a first architecture with a first subset of the set of training data and a second system having a second architecture that is at least in part different from the first architecture with a second subset of the set of training data based on the set of training data to generate, from the first system and the second system, outputs indicating that representing profiles indicate expression of the target proteins based on indicators that are not represented in the set of training data. In at least some examples, the method can include, in response to comparing a first degree of precision of the outputs of the first system to a second degree of precision of the outputs of the second system to determine that the first system or the second system includes an optimized system that has a higher degree of precision for generating the outputs indicating the expression of the target proteins, configuring, by the one or more processors, the system to: select the optimized system from among the plurality of systems based on input of data associated with a different profile in response to comparing the first degree of precision and the second degree of precision; and for at least one different treatment profile, generate an indication of a therapy response based on the optimized system and an output generated by the optimized system.

In some aspects, generating the set of training data can include executing, by the one or more processors, a supervised feature selection algorithm or an unsupervised feature selection algorithm in accordance with the first set of data to identify the one or more indicator types correlated with expression of the target proteins. The method can include selecting, by the one or more processors, the one or more indicator types in response to executing the supervised feature selection algorithm or the unsupervised feature selection algorithm.

In aspects, generating the set of training data can include calculating, by the one or more processors, a feature importance estimate for each indicator of the plurality of indicator types. The method can include selecting, by the one or more processors, the one or more indicator types based on the feature importance estimate for each indicator of the plurality of indicator types.

In at least some aspects, selecting the one or more indicator types can include comparing, by the one or more processors, the feature importance estimate for each indicator of the plurality of indicator types to a threshold value to determine that the one or more indicator types satisfy the threshold value. The method can include selecting, by the one or more processors, the one or more indicator types based on the feature importance estimate satisfying the threshold value.

In some aspects, training the first system and the second system can include training, by the one or more processors, the first system, where the first system includes a first tree-based model that is trained in accordance with gradient boosting techniques. The method can include training, by the one or more processors, the second system, where the second system including a logistic regression model or a penalized logistic regression model.

In aspects, the first system can include an ensemble of models, and training the first system or the second system can include training, by the one or more processors, the ensemble of models to generate the outputs indicative of probabilities that individuals express a target protein correlated with a condition.

In at least some aspects, the one or more indicator types can include one or more first indicator types. Generating the set of training data can include calculating, by the one or more processors, an indicator importance estimate for each indicator of the plurality of indicator types. In examples, the method can include selecting, by the one or more processors, the one or more first indicator types based on the indicator importance estimate for each indicator of the plurality of indicator types. In response to comparing the first degree of precision of the outputs of the first system and the second degree of precision of the outputs of the second system, the method can include determining, by the one or more processors, that the first system outperforms the second system based on a comparison of the first degree of precision with the second degree of precision. In examples, the method can include updating, by the one or more processors, the first system based on the one or more first indicator types and one or more second indicator types from among the plurality of indicator types.

In some aspects, one or more non-transitory computer-readable mediums having instructions stored thereon are described. The instructions, when executed by one or more processors, cause the one or more processors to perform operations including: obtaining a first set of data associated with a plurality of profiles, each profile including a plurality of indicators for a plurality of indicator types. The instructions can cause the one or more processors to generate a set of training data by determining one or more indicator types for one or more indicators that are correlated with expression of target proteins. In examples, the instructions can cause the one or more processors to train a first system having a first architecture with a first subset of the set of training data and a second system having a second architecture that is at least in part different from the first architecture with a second subset of the set of training data based on the set of training data to generate, from the first system and the second system, outputs indicating that representing profiles indicate expression of the target proteins based on indicators that are not represented in the set of training data. In response to comparing a first degree of precision of the outputs of the first system to a second degree of precision of the outputs of the second system to determine that the first system or the second system includes an optimized system that has a higher degree of precision for generating the outputs indicating the expression of the target proteins, the instructions can cause the one or more processors to configure the system to: select the optimized system from among the plurality of systems based on input of data associated with a different profile in response to comparing the first degree of precision and the second degree of precision; and for at least one different treatment profile, generate an indication of a therapy response based on the optimized system and an output generated by the optimized system.

In some aspects, the instructions that cause the one or more processors to generate the set of training data can cause the one or more processors to execute a supervised feature selection algorithm or an unsupervised feature selection algorithm in accordance with the first set of data to identify the one or more indicator types correlated with expression of the target proteins. The instructions can cause the one or more processors to select the one or more indicator types in response to executing the supervised feature selection algorithm or the unsupervised feature selection algorithm.

In aspects, the instructions that cause the one or more processors to generate the set of training data can cause the one or more processors to calculate a feature importance estimate for each indicator of the plurality of indicator types. The instructions can cause the one or more processors to select the one or more indicator types based on the feature importance estimate for each indicator of the plurality of indicator types.

In at least some aspects, the instructions that cause the one or more processors to select the one or more indicator types can cause the one or more processors to compare the feature importance estimate for each indicator of the plurality of indicator types to a threshold value to determine that the one or more indicator types satisfy the threshold value. The instructions can cause the one or more processors to select the one or more indicator types based on the feature importance estimate satisfying the threshold value.

In some aspects, the instructions that cause the one or more processors to train the first system and the second system can cause the one or more processors to train the first system, where the first system includes a first tree-based model that is trained in accordance with gradient boosting techniques. The instructions can cause the one or more processors to train the second system, where the second system including a logistic regression model or a penalized logistic regression model.

In aspects, the first system can include an ensemble of models, and the instructions that cause the one or more processors to train the first system or the second system can cause the one or more processors to train the ensemble of models to generate the outputs indicative of probabilities that individuals express a target protein correlated with a condition.

By virtue of implementation of the techniques described herein, systems can be configured to, at least in part, apply machine learning-based models to predict a probability indicating whether an individual diagnosed as having a disease will live to a certain point after administration of an agent (e.g., a drug, therapeutic, etc.). More specifically, the techniques described herein allow systems to process large, multidimensional datasets that capturing complex interactions and subtle patterns in patient and treatment data that traditional diagnostic systems may not be configured to detect. These models can subsequently allow for personalized medicine by predicting treatment outcomes based on individual patient profiles, thereby minimizing the application of ineffective treatments.

It is to be understood that both the foregoing general description and the following detailed description are exemplary and explanatory, and are intended to provide further explanation of the embodiments described herein.

### BRIEF DESCRIPTION OF THE DRAWINGS

The accompanying drawings constitute a part of this specification, illustrate one or more embodiments and, together with the specification, explain the subject matter of the disclosure.

FIG. 1 is a block diagram of an environment in which one or more devices operate to analyze patient data, in accordance with one or more embodiments described herein.

FIG. 2 is a flow diagram illustrating operations of a method for predicting indicators correlated with a disease in response to analyzing multiple treatment profiles, in accordance with one or more embodiments described herein.

FIGS. 3A-3D are diagrams of example implementations of the method of FIG. 2, in accordance with one or more embodiments described herein.

FIG. 4 is a flow diagram illustrating operations of a method for modeling responses based on administration of an agent, in accordance with one or more embodiments described herein.

FIGS. 5A-5C is a diagram of an example implementation of a process for modeling responses based on administration of an agent, in accordance with one or more embodiments described herein.

### DETAILED DESCRIPTION

Reference will now be made to the embodiments illustrated in the drawings, and specific language will be used here to describe the same. It will nevertheless be understood that no limitation of the scope of the disclosure is thereby intended. Alterations and further modifications of the features illustrated here, and additional applications of the principles as illustrated here, which would occur to a person skilled in the relevant art and having possession of this disclosure, are to be considered within the scope of the disclosure.

Embodiments of the present disclosure relate to systems and methods for determining system performance to select a system having improved accuracy over another system. For example, systems can include one or more processors configured to perform operations to determine a model or group of models that alone, or collectively, outperform one or more other models. In one example, a system can be configured to obtain a first set of individual data associated with a plurality of individuals (e.g., patients or others diagnosed as having a disease as described herein), where a plurality of treatment profiles are correlated with the plurality of individuals. The plurality of treatment profiles indicating indicator (e.g., biomarker) expression levels for a plurality of indicators. The one or more processors can be configured to generate a set of training data by determining, from the first set of individual data, one or more features from among a plurality of features that are correlated with expression of a target protein. In examples, the one or more processors can be configured to train a first model and a second model based on the plurality of treatment profiles in accordance with the one or more features, where training configures the first model and the second model to generate outputs indicative of probabilities that individuals express the target protein. And in response to comparing a first degree of precision of the outputs of a first prediction architecture of the first model and a second degree of precision of the outputs of a second prediction architecture of the second model, the one or more processors can be configured to generate an instruction to execute the first model or the second model having a higher degree of precision to configure a system to analyze a second set of individual data.

By virtue of the implementation of the techniques described herein, processor and memory resource consumption can be reduced by a system trained and configured as described herein. For example, a system can determine one or more features from among a plurality of features that are correlated with expression of a target protein from a first set of individual data and then train models based on a reduced feature set. By having the one or more processors operate on a narrowed set of correlated features rather than the complete set of possible features, model training and inference consume fewer processor cycles and memory allocations than conventional approaches that process all available data dimensions. As a result, the system can perform model selection, training, and inference more efficiently while conserving computational resources without diminishing predictive performance.

In some examples, the techniques described herein can reduce network communication for a system implementing the claimed steps. For example, by generating training data from locally obtained individual data and treatment profiles, and by configuring the one or more processors to compare degrees of precision between locally trained models, the system can avoid transmitting raw high-dimensional datasets to remote systems for analysis. By comparing the outputs of the first model and second model internally and executing the higher precision model within the system, the processes can be completed without multiple communications of full datasets across a network. As a result, overall network traffic is reduced because only minimal instructions or precision metrics are transmitted externally rather than large volumes of raw or intermediate data.

In at least some examples, the system can improve accuracy when compared to other systems by selectively executing the model having a higher degree of precision as determined. For example, by training the first model and the second model on the plurality of treatment profiles using the features correlated with expression of the target protein, systems can confirm that each model is optimized for relevant and discriminative data elements. By comparing the first degree of precision of the outputs of the first prediction architecture with the second degree of precision of the outputs of the second prediction architecture, the system can identify which model(s) more accurately predict target protein expression for given inputs. As a result, deployment of the higher precision model allows the system to consistently generate more accurate outputs than systems that do not perform model comparison and selection based on precision metrics.

Embodiments of the present disclosure also relate to systems and methods for determining system performance for a plurality of systems to select a system having improved accuracy over another system when determining differences between protected profiles (e.g., treatment profiles and/or limited treatment profiles as described herein). For example, a system can include one or more processors. The one or more processors can be configured to obtain a first dataset comprising a first plurality of elements that correspond to treatment profiles of individuals, where each treatment profile comprises indicators and corresponding values that quantify a state of an individual. In examples, the one or more processors can be configured to determine a set of features from among a plurality of features based on a subset of elements from among the plurality of elements. The one or more processors can be configured to generate an analysis dataset comprising a second plurality of elements based on the first dataset and the set of features, the second plurality of elements corresponding to treatment profiles that at least in part satisfy the set of features. In some examples, the one or more processors can be configured to train a plurality of models based on the analysis dataset, where performance of each model of the plurality of models when determining a time-to-event prediction is evaluated using a different subset of the analysis dataset. The one or more processors can then determine, for each individual represented by the subset of elements, a plurality of performance metrics using each model of the plurality of models. In examples, the one or more processors can determine, for each individual represented by the subset of elements, an administration response based on the plurality of performance metrics corresponding to each individual.

By virtue of the implementation of the techniques described herein, processor and memory resource consumption can be reduced by a system configured to select and execute models when determining differences between protected profiles. For example, the system can determine a set of features from among a plurality of features based on a subset of elements from a first dataset, and generate an analysis dataset comprising only treatment profiles that at least in part satisfy the set of features (e.g., include usable values or values that can be imputed based on other values obtained for the represented individuals). By reducing the dataset to only relevant elements and training models using this reduced analysis dataset, the one or more processors can operate on smaller data structures and avoid repetitive computation associated with irrelevant features or unfiltered profiles. As a result, the system can conserve processor cycles and memory allocations while maintaining analytical relevance for time-to-event predictions.

In examples, a system operating as described herein can improve accuracy when compared to other systems that do not perform such targeted feature selection and multi-model and/or multi-configuration evaluation. For example, by determining a set of features from the plurality of features that are most relevant to a time-to-event prediction, the models are trained on inputs that have stronger predictive relationships to the outcome. By evaluating the performance of each model using distinct subsets of the analysis dataset and deriving administration responses from the performance metrics corresponding to each individual, the system can confirm that the selected model delivers more accurate and personalized predictions. As a result, the overall predictive performance improves, with the system producing outputs that more reliably reflect true clinical timelines than systems lacking adaptive feature selection and multi-model comparison.

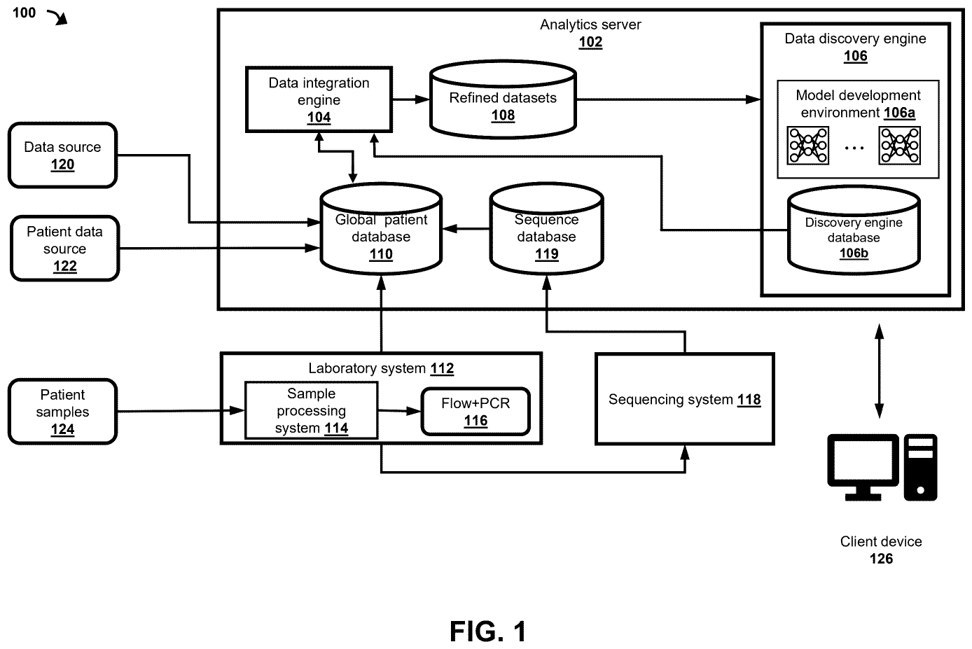

FIG. 1 is a block diagram of an environment 100 for analyzing patient data, according to an embodiment. The environment 100 can include an analytics server 102, a laboratory system 112, a sequencing system 118, a data source 120, patient data source 122, patient samples 124, and a client device 126. Various components depicted in FIG. 1 can belong to an organization involved in clinical research of one or more diseases such as, for example, acute myeloid leukemia (AML) or other diseases and/or to one or more organizations involved in treating patients with the one or more diseases. While certain components and devices are illustrated as being included in the environment 100 of FIG. 1, it will be understood that the environment 100 is not confined to the components or diseases as described herein and can include additional or different components (not shown for purposes of brevity and clarity) which are configured to be considered within the scope of the embodiments described herein.

In some embodiments, the analytics server 102 can include any computing device comprising a processor and non-transitory machine-readable storage capable of executing the various tasks, processes, and/or operations as described herein. The analytics server 102 can employ various processors such as central processing units (CPUs), graphical processing units (GPUs), and/or the like. Some non-limiting examples of such computing devices can include workstation computers, laptop computers, server computers, and/or the like. While the environment 100 includes a single analytics server 102, there can be multiple analytics servers 102. Further, the analytics server 102 can include any number of computing devices operating in a distributed computing environment such as, for example, a cloud computing environment. As described herein, the analytics server 102 can include a data integration engine 104, a data discovery engine 106, refined datasets 108, a global patient database 110, and a sequence database 119. In some embodiments, the analytics server 102 can include and/or implement operations that are associated with the laboratory system 112, the sequencing system 118, and/or the client device 126. In some embodiments, the analytics server 102 can include and/or implement operations that are associated with (e.g., involved in the generation of) the data source 120, the patient data source 122, and/or the patient samples 124.

In some embodiments, the analytics server 102 can be configured to receive data from the data source 120, the patient data source 122, and the laboratory system 112 and sequencing system 118 when processing patient samples 124. For example, the analytics server 102 can be configured to receive data from the data source 120, where the data is associated with (e.g., represents) entries corresponding to one or more patient files. As an example, as patients interact with clinicians, the clinicians can generate information that is received as input at a client device (not explicitly illustrated) that is associated with the clinicians, the notes indicating clinical observations and/or updates to treatment plans for the patients made by the clinician. The client device can then generate patient data that is associated with each patient and representative of the clinical observations or updates to the treatment plans and store the patient data in the data source 120 to later transmit to the analytics server 102. In this example, the analytics server 102 can implement the global patient database 110 such that the patient data is uploaded and stored in the global patient database 110 in association with one or more identifiers for the patient as described herein.

In another example, the analytics server 102 can be configured to receive data from the patient data source 122, where the data is associated with (e.g., represents) information about individual patients. As an example, as a history of a patient is obtained, the clinicians and/or the patients can generate information that is received as input at a client device (not explicitly illustrated) that is associated with the clinicians and/or patients, the information indicating aspects of the history of the patient such as whether the patient is associated with a history of a given disease in their family, whether the patient had any exposure to environmental conditions associated with the given disease, and/or the like. The client device can then generate patient data that is associated with each patient and representative of the history of the patient and store the patient data in the patient data source 122 to later transmit to the analytics server 102. In this example, the analytics server 102 can obtain and store the patient data in the global patient database 110 in association with one or more identifiers for the patient as described herein.

In yet another example, the analytics server 102 can be configured to receive data from the laboratory system 112 and/or the sequencing system 118, where the data is associated with (e.g., represents) information about patient samples (e.g., tissue samples, blood samples, blood counts (e.g., complete blood counts), bone marrow aspiration and biopsy results, lumbar puncture results, and/or the like) as well as the results of the processing of the samples (e.g., a DNA sequence or targets thereof). As an example, as a patient is evaluated and/or treated for a disease such as AML, patient samples 124 similar to those described above can be obtained. The patient samples 124 can be initially obtained and processed by a laboratory system 112 and processed by a sample processing system 114. The sample processing system 114 can implement one or more devices configured to obtain and store the patient samples and extract DNA from the patient samples. For example, in preparation for genetic analysis to guide AML treatment, patient blood or bone marrow can first be obtained from a patient and frozen. Later, these samples can be quality checked to ensure the sample purity and quantity are sufficient for sequencing. In some embodiments, the isolated DNA can then undergo further processing to be separated into manageable fragments and equipped with adapters (e.g., short, specific pieces of synthetic DNA associated with the fragmented DNA molecules) for compatibility with sequencing machines. In some embodiments, the samples can also be provided to a flow and polymerase chain reaction (PCR) system to extract and amplify the isolated DNA. The laboratory system 112 can then provide the processed samples and corresponding data representing the samples to be processed by the sequencing system 118. Additionally, or alternatively, the laboratory system 112 can then provide the data generated by the laboratory system 112 when processing the samples to the analytics server 102 to be stored in the global patient database 110.

In some embodiments, the sequencing system 118 can be configured to receive the patient samples and/or the isolated DNA and sequence the patient samples. In one example, the sequencing system 118 can attach DNA fragments to a surface in a specific pattern, creating clusters. The sequencing itself can involve a series of cycles where fluorescently labeled nucleotides are introduced one by one. The incorporation of each base can be detected, identifying the sequence of the fragment base by base. Finally, the sequencing system 118 can analyze the vast amount of data, assemble the original DNA sequences and identify any variations or mutations present (sometimes referred to as Next-Generation Sequencing (NGS)). The sequencing system 118 can then provide data associated with the sequenced DNA to the analytics server 102. In this example, the analytics server 102 can store the sequenced DNA in a sequence database 119 that stores the sequenced DNA in association with one or more profile identifiers established by the analytics server 102. In some embodiments, the analytics server 102 can also cause the sequence database 119 to provide the data associated with the sequenced DNA to the global patient database 110 to be stored in association with other data associated with the patient such as a treatment profile and/or limited treatment profile for the patient as described herein.

In some embodiments, the analytics server 102 can implement a data integration engine 104 to process data stored in the global patient database 110. For example, the analytics server 102 can implement the data integration engine 104 such that the data integration engine 104 is configured to obtain the data associated with the patients that is stored in the global patient database 110 and processes the data to be used by the data discovery engine 106. In one example, as data is obtained by the global patient database 110 for a given patient, the data can be stored in the global patient database 110 in association with one or more identifiers as part of a profile for the patient. The data integration engine 104 can then obtain the data associated with the patient (e.g., the entire profile or portions thereof) from the global patient database 110 and process the data to generate a limited treatment profile. The limited treatment profile can then be stored in the refined datasets database 108 (referred to herein as “refined datasets”) and made available to the data discovery engine 106. In this way, the analytics server 102 can maintain two separate datasets that allow for updates to the limited treatment profiles stored in the refined datasets 108 and subsequent use by the data discovery engine 106 when performing the operations described herein. As will be understood, in this example the data associated with the patient that is stored in the global patient database 110 can be updated over time such that the patient profile is represented as a set of entries associated with a time series. As the global patient database 110 is updated, the data integration engine 104 can obtain updated versions of the data associated with the patient from the global patient database 110, process the data when updating the limited treatment profiles in the refined datasets 108, and store the updates in the refined datasets 108.

In some embodiments, the analytics server 102 can implement the data discovery engine 106 that includes a model development environment 106a and a discovery engine database 106b. For example, the analytics server 102 can implement the data discovery engine 106 such that the data discovery engine 106 is configured to receive data associated with one or more limited treatment profiles that are stored in the refined datasets 108 and process the one or more limited treatment profiles. In this example, the analytics server 102 can process the one or more limited treatment profiles using the model development environment 106a. Processing the limited treatment profiles can include providing the limited treatment profiles to one or more models (e.g., machine learning-based models, including supervised models such as linear regression models and unsupervised models such as clustering models, and/or the like) to determine one or more metrics. The one or more metrics can represent the performance of each of the models, indicating which model or groups of models are more or less accurate, efficient, and/or the like at generating one or more predictions compared to one or more other models. These predictions can include indications of treatment options that have a likelihood of optimizing an outcome (e.g., life extension) for the patients. In some embodiments, the analytics server 102 can process the limited treatment profiles to determining one or more aspects of the limited treatment profiles. For example, where the limited treatment profile is associated with a predetermined number of possible attributes but the patient samples 124 that are available are limited and only usable to determine a subset of the possible attributes, the model development environment 106a can process the portions of the refined patient profile that are available in the refined datasets 108 to determine one or more of the remaining attributes of the possible attributes. In this example, data associated with the one or more remaining attributes can be stored by the data discovery engine 106 in the discovery engine database 106b along with an identifier from the limited treatment profile (e.g. the pseudo-identifier and/or other entries in the limited treatment file). The analytics server 102 can then periodically or in real-time update the global patient database 110 based on the data associated with the limited treatment profiles (e.g., the one or more remaining attributes and/or the like) that are stored in the discovery engine database 106b.

In some embodiments, the data associated with the one or more remaining attributes can be transmitted by the discovery engine database 106b to the data integration engine 104. The data integration engine 104 can identify which treatment profile and/or limited treatment profile the data associated with the one or more remaining attributes corresponds to based on the identifier. In some embodiments, the data integration engine 104 can then update the treatment profile and/or the limited treatment profile in accordance with the one or more remaining attributes. For example, the data integration engine 104 can update the treatment profile and/or the limited treatment profile with an indication of the appropriate treatment (e.g., that is predicted to optimize the lifespan of the patient) based on analysis of the entries of the treatment profile and/or limited treatment profile. In an example, the data integration engine can access the global patient database 110 and update the entries of the treatment profile in accordance with the remaining attributes. In another example, the data integration engine 104 can access the refined datasets 108 and update the entries of the limited treatment profile in accordance with the remaining attributes.

In some embodiments, the client device 126 can include any computing device comprising a processor and non-transitory machine-readable storage capable of executing the various tasks, processes, and/or operations as described herein. The client device 126 can employ various processors such as central processing units (CPUs), graphical processing units (GPUs), and/or the like. Some non-limiting examples of such computing devices can include workstation computers, laptop computers, server computers, and/or the like. While the environment 100 includes a single client device 126, there can be multiple client devices 126. Further, the client device 126 can include any number of computing devices operating in a distributed computing environment such as, for example, a cloud computing environment. In some embodiments, the client device 126 can be associated with one or more software developers and/or one or more clinicians that are interacting with (e.g., configuring operation of) the analytics server 102 as described herein. In some embodiments, the client device 126 can be associated with one or more clinicians and/or one or more organizations involved in treating patients with the one or more diseases such as a hospital and/or the like.

In some embodiments, the analytics server 102 can generate and display an electronic platform (e.g., via the client device 126) when receiving and processing patient data associated with one or more patients, performing one or more operations when analyzing the patient data, and outputting data associated with the results of the operations performed by any of the components of the analytics server 102 such as, for example, the data discovery engine 106. The electronic platform can include graphical user interfaces (GUI) displayed by display devices of one or more client devices 126. An example of the electronic platform generated and hosted by the analytics server 102 can be a web-based application or a website configured to be displayed on different electronic devices, such as mobile devices, tablets, personal computers, and the like.

In some embodiments, treatment profiles and/or limited treatment profiles may be analyzed to identify trends, commonalities, and divergences across patients or patient subgroups. Such analysis can include direct comparison of temporal treatment sequences, cumulative dosing exposures, treatment intensities, or intervals between successive interventions. By evaluating these patterns, clinicians and researchers may discern which specific treatment pathways or regimen characteristics are consistently associated with improved or diminished outcomes, the analytics server 102 can execute one or more operations to assist with clinical decision-making. In certain cases, composite measures derived from the treatment profiles (e.g., such as dose-density indices, treatment adherence scores, or timing-of-intervention metrics) can be calculated and examined to assess their relationship to patient outcomes. The analysis may additionally include the use of statistical or machine learning algorithms to identify correlations between specific intervention sequences, dosing regimens, or therapeutic combinations and one or more clinical outcome metrics. Such analysis may involve aggregating patient-level treatment history data, mapping these histories against measured outcomes such as overall survival, event-free survival, progression-free survival, or response rates, and applying predictive modeling to determine which profile features are most strongly associated with favorable clinical endpoints. The resulting output generated by the analytics server 102, represented by the treatment profiles, can be used to generate user interfaces that can be displayed (e.g., at the client device 126) to indicate therapies to administer and/or allow for personalized treatment recommendations, optimize protocol design, or adjust ongoing therapy, thereby improving patient prognosis and enhancing resource utilization in clinical practice.

The above-mentioned components can be configured to interconnect with to each other and establish communication connections therebetween through a network (not explicitly illustrated). Examples of the network can include, but are not limited to, private or public local-area-networks (LAN), wireless LAN (WLAN) networks, metropolitan area networks (MAN), wide-area networks (WAN), and the Internet. The network can include wired and/or wireless communications according to one or more standards and/or via one or more transport mediums. The communication over the network can be performed in accordance with various communication protocols such as Transmission Control Protocol and Internet Protocol (TCP/IP), User Datagram Protocol (UDP), and IEEE communication protocols. In one example, the network can include wireless communications according to Bluetooth specification sets or another standard or proprietary wireless communication protocol. In another example, the network can also include communications over a cellular network, including, e.g., a GSM (Global System for Mobile Communications), CDMA (Code Division Multiple Access), and EDGE (Enhanced Data for Global Evolution) network.

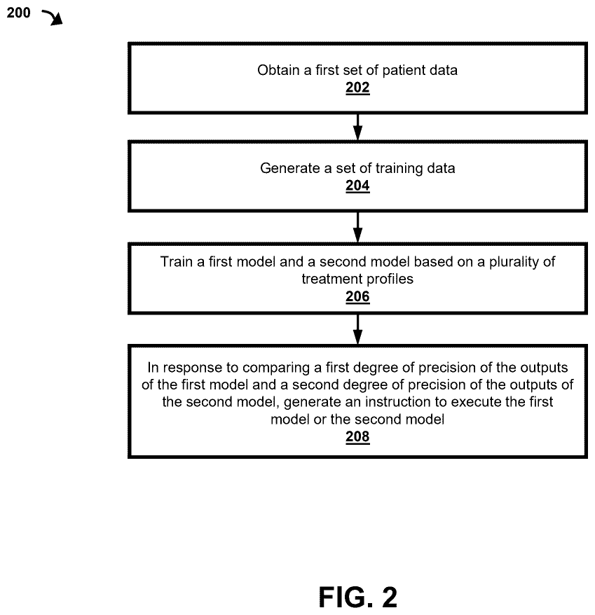

FIG. 2 is a flow diagram illustrating operations of a method 200 for predicting indicators correlated with a disease in response to analyzing multiple treatment profiles, in accordance with one or more embodiments described herein. In some implementations, one or more of the functions described with respect to the method 200 can be performed (e.g., completely, partially, and/or the like) by an analytics server (or one or more components thereof such as a model development environment) that is the same as, or similar to, the analytics server 102 of FIG. 1. In some implementations, one or more of the functions described with respect to the method 200 can be performed (e.g., completely, partially, and/or the like) by another device or group of devices separate from and/or including the analytics server, such as by one or more client devices that are the same as, or similar to, the client device 126 of FIG. 1.

At operation 202, the analytics server can obtain a first set of patient data associated with a plurality of patients. For example, the analytics server can obtain the first set of patient data where the first set of patient data is associated with (e.g., includes and/or represents) treatment profiles and/or limited treatment profiles as described herein that each correspond to entries (also referred to as elements of the patient data and/or corresponding dataset) for patients of the plurality of patients. In this example, each treatment profile can include one or more entries corresponding to one or more indicators (also referred to as biomarkers). In some embodiments, the treatment profiles can additionally, or alternatively, include entries indicating bibliographic data associated with bibliographic information about the one or more patients (e.g. name, date of birth, address, family history of the one or more diseases (or related diseases), and/or the like). In some examples, the bibliographic information can represent a medical history of the patient (e.g., diagnosis of the one or more diseases, date(s) of diagnosis, treatment(s), date(s) of treatment, and/or the like). In examples, the treatment profiles can include entries representing measurements for one or more of the biomarkers measured for the respective patient at one or more points in time. While certain aspects of the present disclosure are described with respect to particular diseases (e.g., AML) it will be understood that the present disclosure is not limited to such diseases and that the presently-described techniques can be applied to predicting indicators correlated with a disease.

In some embodiments, the analytics server can obtain the first set of patient data from one or more client devices that are controlled by one or more clinicians. For example, a client device can receive input from a clinician indicative of the bibliographic information about the patient, generate at least a portion of the first set of patient data, and provide the first set of patient data to the analytics server. In these examples, the analytics server can store the first set of patient data (also referred to as a first set of individual data) in a global patient database (e.g., that is the same as, or similar to, the global patient database 110 of FIG. 1) based on receiving the patient data. In another example, the client device can be controlled by a clinician in coordination with execution of one or more operations by a laboratory system and/or a sequencing system. In this example, the client device can be included in and/or implemented by the laboratory system and/or the sequencing system and configured to process and generate data associated with patient samples corresponding to the plurality of patients. The client device can then provide the data associated with the patient samples to the analytics server to be included in the treatment profiles and/or the limited treatment profiles. In one example, where a patient is suspected of having, or was previously diagnosed as having, a given disease such as AML, the client device can obtain data from the laboratory system and/or the sequencing system representing one or more biomarkers and/or expression levels for the biomarkers and provide the data to the analytics server.

At operation 204, the analytics server can generate a set of training data. For example, the analytics server can generate a set of training data based on data represented by the plurality of treatment profiles. In some embodiments, the set of training data can include treatment profiles (and/or updated versions of the treatment profiles) that represent feature values for one or more features correlated with one or more biomarkers as indicative of the disease. In one example, the set of training data can include treatment profiles that include feature values representing biomarker measurements that are correlated with expression of a target protein (e.g., CD70) that is indicative of the disease. In this example, the target protein can be a protein whose expression beyond a threshold value is predetermined to be indicative of the disease.

In some embodiments, the analytics server can determine the one or more features from among a plurality of features that are correlated with the disease. For example, the analytics server can execute a supervised feature selection algorithm or an unsupervised feature selection algorithm in accordance with the patient data to identify the one or more features correlated with expression of the target protein. In examples where the analytics server is executing a supervised feature selection algorithm or an unsupervised feature selection algorithm when determining features indicative of AML, the analytics server can do so to identify one or more features such as biomarkers and corresponding marker values that are correlated with the expression of CD70 at a level that can indicate the presence of AML.

The supervised feature selection algorithm can be configured to identify features (e.g., a different biomarker or combination of biomarkers other than the target protein) that are indicative of the disease and/or patient outcomes for patients that are diagnosed as having the disease based on a relationship between the features and the target protein. Examples of supervised feature selection algorithms can include filter methods (e.g., Pearson or Spearman for assessing linear and monotonic relationships, the Chi-squared test for categorical data to check independence from the class) which score features based on statistical tests, wrapper methods (e.g., forward selection, backward elimination, recursive feature elimination, etc.) which evaluate subsets of features by iteratively training models and comparing their ability to accurately predict patients as having the disease, and embedded methods (e.g., by virtue of the implementation of decision trees, random forests, etc.) where feature selection is integrated into the model training itself. The analytics server can then calculate a feature importance estimate for each feature of the plurality of features available in the treatment profiles based on the relationship between the feature and the expression of the target protein that is indicative of the disease. The feature importance estimate can be represented as a value (e.g., between 0 and 1 or any other suitable range of values) to indicate a correlation between the feature and the expression of the target protein.

The unsupervised feature selection algorithm can be configured to determine feature importance based on intrinsic properties of the features or their relationships to the target protein within the dataset. For example, the analytics server can determine a feature importance estimate by evaluating factors such as the contribution of a given feature represented in the treatment profiles to reconstruction errors in, for example, autoencoders fit to the treatment profiles. In this example, the analytics can attribute a higher feature importance estimate to features where error increases beyond a threshold amount upon feature removal of the feature.

In some embodiments, the analytics server can determine the one or more features from among a plurality of features by selecting the one or more features based on their feature importance estimate. For example, the analytics server can compare the feature importance estimate for each feature of the plurality of features to a threshold value to determine that the one or more features satisfy the threshold value. In this example, the threshold value can be a predetermined value (e.g., a value within a range established by the feature importance estimates corresponding to each feature represented by the treatment profiles). In another example, the threshold value can indicate a percentile that divides the plurality of features (e.g., the plurality of biomarkers) according to their relative correlation to the target protein. The analytics server can then select the one or more features based on the importance estimates and the threshold value(s).

In some embodiments, the analytics server can determine one or more sets of features to be fit to one or more models, where each set of features are associated with similar feature importance estimates. For example, the analytics server can first determine feature importance estimates for the features represented by the treatment profiles as described herein. The analytics server can then compare the feature importance estimates to determine one or more groupings. For example, the analytics server can determine a group of one or more first features that are associated with a percentile range (e.g., the 90th percentile). The analytics server can then determine a group of one or more second features that are associated with a different percentile range (e.g., the 80th to 90th percentile, etc.). The analytics server can then select the one or more features based on the groupings such that the features selected include and/or exclude the first features and/or the second features.

At operation 206, the analytics server can execute (e.g., train and/or fit) a first model and a second model based on a plurality of treatment profiles. For example, the analytics server can execute the first model and the second model to configure each model to predict a likelihood that a given patient is associated with a given biomarker and corresponding marker value. In some examples, the analytics server can train the first model and the second model based on the plurality of treatment profiles in accordance with the one or more features that were selected to predict a likelihood that the target protein indicative of the disease is expressed by the patient in accordance with the selected features (described above). As described herein, the analytics server can train a plurality of models (e.g., a first model, a second model, a third model, etc.) using the features available in the treatment profiles, where each model of the plurality of models has a different architecture (e.g., a first prediction architecture, a second prediction architecture, etc.). Additionally, or alternatively, one or more models having similar or different architectures can be combined (e.g., as an ensemble) and trained. As a result, the first model and the second model can be configured to receive feature values from treatment profiles corresponding to the selected features (e.g., that do not directly indicate an expression level of the target protein) and predict a likelihood that the target protein is expressed by the patient (e.g., that the expression level of the protein satisfies a threshold expression value) based on these feature values.

In examples, training the first model or the second model can involve training a tree-based model. For example, the analytics server can train a decision tree, a random forest, etc. using the training dataset. During training of the first model and/or the second model, the analytics server can construct a decision tree where each node represents a binary decision based on features from the treatment profiles included in the training data. In one example, a node in a decision tree can represent a decision about whether a biomarker is represented by a given treatment profile and/or that the marker value for that biomarker as represented by the treatment profile satisfies a threshold value established for that biomarker by the node. The analytics server can implement an algorithm like CART (Classification And Regression Trees) to recursively split the data at each node for classification (e.g., classification of each treatment profile as being associated with expression of the target protein). Where the first model or the second model includes a tree-based model such as a random forest, the analytics server can further build multiple, different trees and aggregate the prediction of each tree to determine the probability that the treatment profile is associated with expression of the target protein.

In some examples, training the first model or the second model can involve training one or more artificial neural networks (ANNs). These ANNs can include, feed-forward neural networks, convolutional neural networks, tree-based models (e.g., decision trees) that are trained using ensemble methods like gradient boosting techniques, etc.), and so on. In these examples, the analytics server can provide the plurality of treatment profiles to each model to cause the models to generate corresponding outputs. These outputs can include probabilities that the measured biomarkers included in a given treatment profile are indicative of whether respective patients express the target protein. The analytics server can then compare the probabilities to known probabilities (e.g., established based on the expression level of the target protein as indicated in the treatment profiles) and iteratively update the models by adjusting one or more weights of the models until the predicted probabilities differ from the known probabilities by a threshold value that satisfies an acceptance value (e.g., the model converges).

In examples, the first model or the second model can include a logistic regression model or a penalized logistic regression model. In these examples, the analytics server can train the logistic regression model based on the treatment profiles represented by the training data to predict whether the target protein is expressed. In this example, the analytic server can use the biomarkers represented by the treatment profiles as input features, while the binary expression status of the target protein (e.g., expressed or not expressed) serves as the target variable. The logistic function can be configured to determine a probability that the protein is expressed based on a linear combination of these features. In this example, the analytics server can tune the model parameters by estimated the likelihood function, or equivalently, minimizing the negative log-likelihood (binary cross-entropy loss) using optimization methods like gradient descent. During training, the analytics server can iteratively adjusts the model's coefficients to better predict the expression of the protein until the model converges.

In some embodiments, the analytics server can determine a first degree of precision for the first model and a second degree of precision for the second model. For example, the analytics server can reserve a portion of the training dataset for validation. In this example, the analytics server can execute the models in accordance with the reserved portion of the training dataset to make predictions as to a degree to which the target protein is expressed for each treatment profile in the reserved portion of the training dataset. The analytics server can then calculate a ratio of true positives to the sum of true positives and false positives to determine the accuracy of the positive predictions generated by the first model and the second model, and compare the performance of the two models to determine the first degree of precision and the second degree of precision, respectively. The analytics server can then determine whether the first model outperformed the second model, or vice versa.

In some embodiments, the analytics server can augment the training data during execution of the models to increase the training data and/or introduce variability in the training data. For example, the analytics server can implement a resampling technique (e.g., bootstrapping) to augment the existing treatment profiles. By repeatedly drawing random samples with replacement from the treatment profiles represented by the training data, the analytics server can create multiple simulated datasets of varying size. The analytics server can then evaluate performance of the models and select a model when fit to the varying sets of training data and determine an average degree of precision based on performance of a given model over multiple training iterations. The average degree of precision can then be established as being the degree of precision for that model.

At operation 208, the analytics server can, in response to comparing a first degree of precision of outputs of the first model and a second degree of precision of outputs of the second model, generate an instruction to execute the first model or the second model. For example, the analytics server can generate an instruction to execute the first model or the second model having a higher degree of precision to configure a system (e.g., the client device) to analyze a second set of patient data (also referred to as a second set of individual data). In one example, where the client device is associated with a clinician that is diagnosing patients as having or not having the disease, the analytics server can provide data associated with the first model or the second model (e.g., the outperforming model) to the client device. The data associated with the first model or the second model can be configured to cause the client device to receive data associated with patients being treated by the clinician and determine whether the patients have the disease.

In some embodiments, the analytics server can execute a limited portion of the training dataset to perform one or more of the operations described herein (e.g., operations 202-206). For example, to assess the first model and the second model, the analytics server can determine a subset of data (e.g., a subset of elements/entries/etc.) available to the analytics server to use when executing (e.g., training and/or fitting) the models. Once the analytics server determines that a given model outperformed the other(s), the analytics server can update and/or retrain the selected model on the entire training dataset. In this way, the analytics server can identify a model that is optimized to identify expression of the target protein without consuming computational resources that would otherwise be needed to fully train each model prior to assessment of the models'abilities.

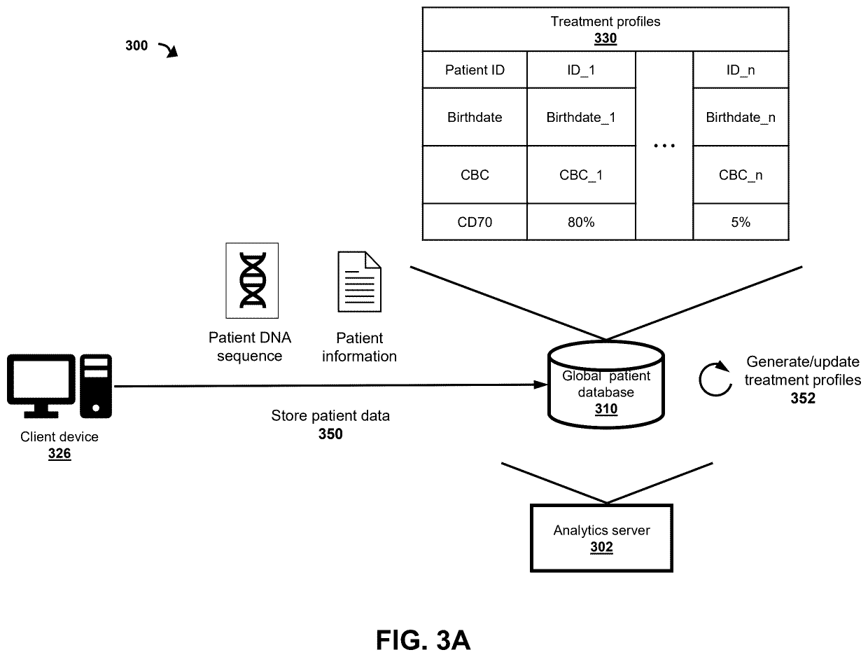

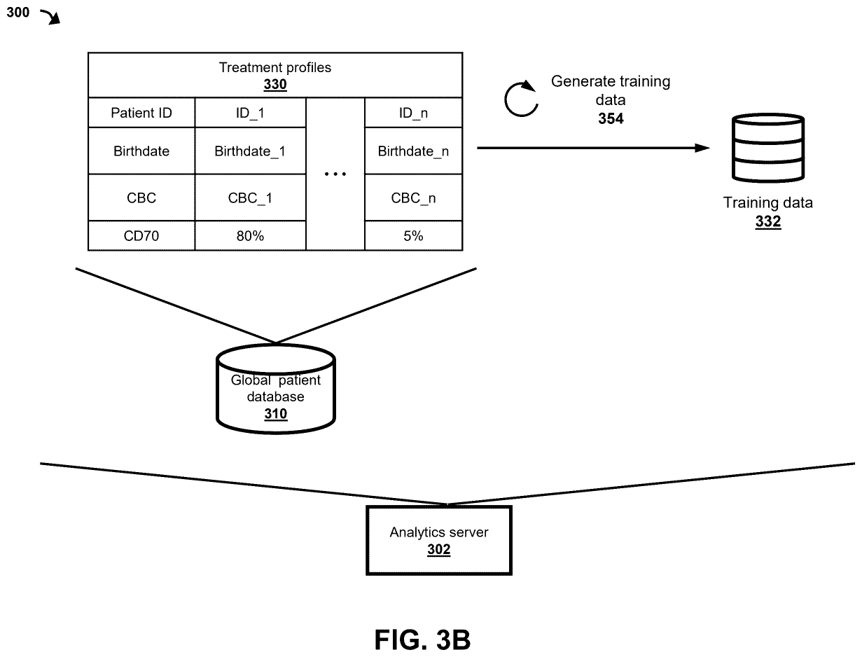

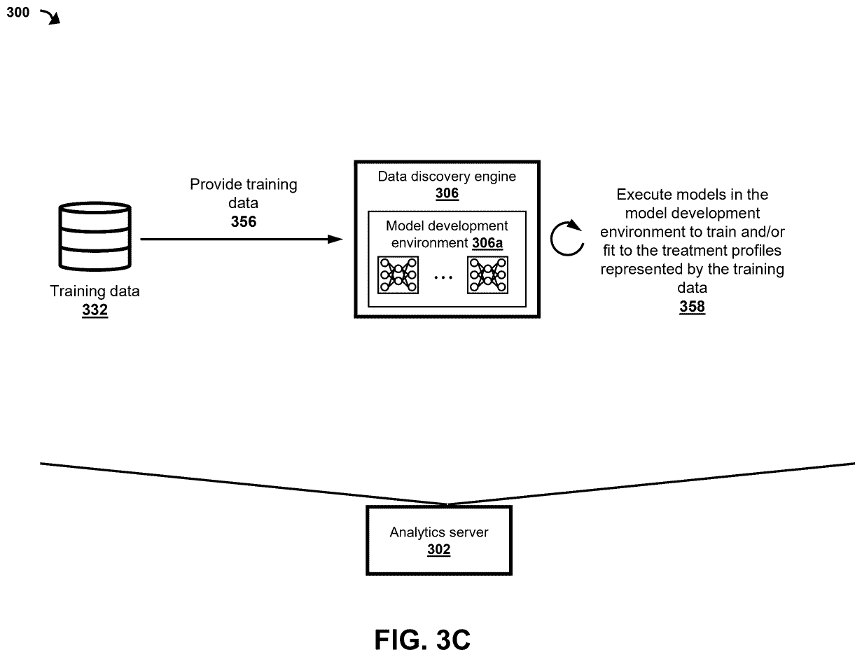

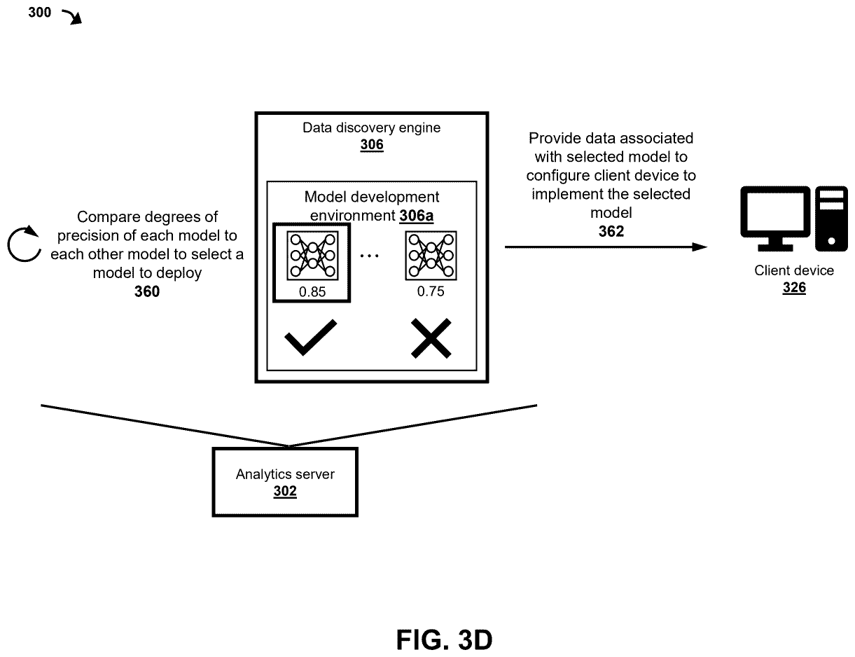

FIGS. 3A-3D are a diagram of an example implementation 300 of the method 200 of FIG. 2, in accordance with one or more embodiments described herein. In some embodiments, the operations of the implementation 300 can be implemented by an analytics server 302, a global patient database 310, a laboratory system 314, a sequencing system 318, and a client device 326 that are the same as, or similar to, the analytics server 102, the global patient database 110, the laboratory system 114, the sequencing system 118, and/or the client device 126 of FIG. 1. Additionally, or alternatively, one or more of the operations of the implementation 300 can involve a data integration engine 304, a global patient database 310 and/or a sequence database 319 that are the same as, or similar to, the data integration engine 104, a global patient database 110 and/or a sequence database 119 of FIG. 1.

At operation 350, the analytics server 302 can obtain a first set of patient data associated with a plurality of patients from a client device 326. For example, the analytics server 302 can receive data associated with measured biomarkers of the plurality of patients over a period of time (e.g., during regularly-scheduled clinician visits, during treatment for a disease such as AML, etc.). The measured biomarkers can include any of the biomarkers described herein, including biomarkers indicating a target protein that is associated with a disease (e.g., in the case of AML, expression of CD70 as determined through flow cytometry testing, etc.). The analytics server 302 can then store the first set of patient data in the global patient database 310 as one or more treatment profiles 330.

At operation 352, the analytics server 302 can generate and/or update a set of treatment profiles 330. For example, in response to receiving the patient data, the analytics server 302 can generate and/or update the treatment profiles 330 such that each treatment profile includes one or more entries indexed according to respective patients. In one example, the entries can include biomarkers that are determined through sample testing of patient samples such as complete blood counts (CBCs), birthdates (indicating age), and expression levels of a target protein (e.g., that 5%, 80%, etc., of the target protein measured relative to a range of expression measurements for that target protein).

At operation 354, the analytics server 302 can generate training data 332 based on the treatment profiles 330. For example, the analytics server 302 can generate the training data in response to determining one or more features from among a plurality of features that are represented by the treatment profiles 330. These can include biomarkers that are correlated with expression of a target protein. In some examples, the analytics server 302 can calculate a feature importance estimate for the biomarkers represented by the treatment profiles 330 as described herein and select the one or more features for generating the training data 332.

At operation 356, the analytics server 302 can provide the training data to a data discovery engine 306 to be executed by a model development environment 306a. For example, the analytics server 302 can provide the training data to cause the model development environment 306a to execute (e.g., train and/or fit) a plurality of models (including at least a first model and a second model). The plurality of models can be associated with different architectures. For example, the plurality of models can include tree-based models (e.g., decision trees, random forests, etc.), ANNs, linear regression models, penalized linear regression models, etc.

At operation 358, the analytics server 302 can execute the models in the model development environment 306a to train and/or fit the models to the treatment profiles represented by the training data. The analytics server 302 can execute the models using a subset of the training dataset, reserving at least a portion of the training dataset for training once a specific model is identified as having a higher degree of precision than the other models. For example, after training and/or fitting the models of the model development environment 306a, the analytics server 302 can determine corresponding degrees of precision indicating the ability of each model to be trained and/or fit to the treatment profiles in the training data.

At operation 360, the analytics server 302 can compare a degree of precision associated with each model to degrees of precision of each other model executed by the model development environment 306a. For example, the analytics server can compare the degrees of precision of the ability of each model to correctly classify sets of biomarkers as being correlated with a target protein indicative of a disease. In one example, a first model can be associated with a first degree of precision (“0.85”) that is greater than a second degree of precision associated with a second model (“0.75”). The analytics server 302 can then select a model from among the plurality of models (e.g., the first model having the first degree of precision) that has a higher degree of precision than one or more other models and train the selected model using the data reserved from the training data.

At operation 362, the analytics server 302 can generate and provide data associated with the selected model to the client device 326 to configure the client device to implement the selected model. For example, the analytics server 302 can generate the data associated with the selected model to configure the client device 326 to execute the selected model using biomarkers that the model is trained and/or fit to analyze. Additionally, the client device 326 can be configured to generate a graphical user interface (GUI) indicating the output of the model. In one example, where an individual is suspected as having AML, the client device 326 can be configured to receive data associated with biomarkers of that individual that the selected model is capable of processing. The client device 326 can then be configured to execute the selected model using the outputs to determine whether or not the biomarkers correlate with the target protein (e.g., CD70) and, as a result, whether the individual has or does not have AML.

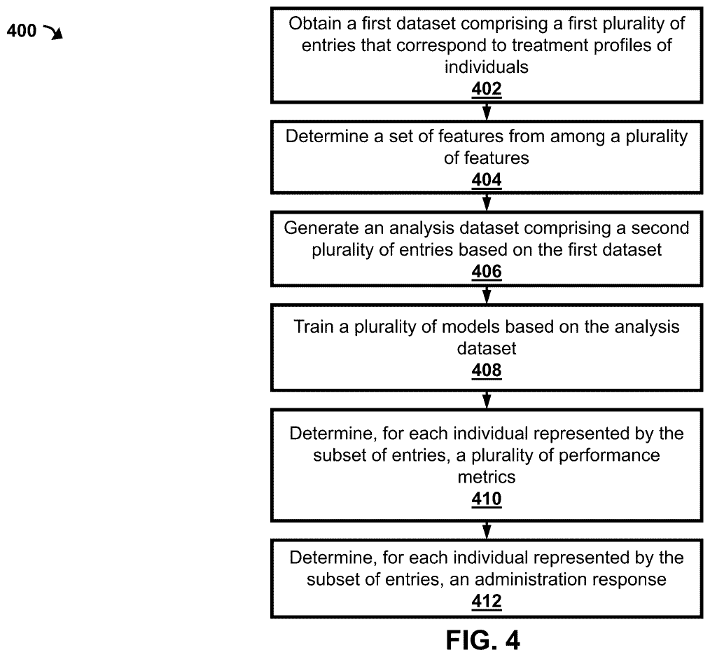

FIG. 4 is a flow diagram illustrating operations of a method 400 for modeling responses based on administration of an agent, in accordance with one or more embodiments described herein. In some implementations, one or more of the functions described with respect to the method 400 can be performed (e.g., completely, partially, and/or the like) by an analytics server that is the same as, or similar to, the analytics server 102 of FIG. 1. In some implementations, one or more of the functions described with respect to the method 400 can be performed (e.g., completely, partially, and/or the like) by another device or group of devices separate from and/or including the analytics server, such as by one or more client devices that are the same as, or similar to, the client device 126 of FIG. 1.

At operation 402, the analytics server can obtain a first dataset comprising a first plurality of entries that correspond to treatment profiles of individuals. For example, the analytics server can obtain a first dataset comprising a first plurality of entries that correspond to treatment profiles of individuals that are diagnosed as having or not having a disease (e.g., AML, etc.). The treatment profiles of the first dataset can include treatment profiles and/or limited treatment profiles as described herein. While the techniques discussed make reference to diseases such as AML, it will be understood that they are not limited to AML and that they can be implemented in view of any suitable combination of disease and agent(s).

In some embodiments, each treatment profile can comprise (e.g., include) indicators such as biomarkers that are represented as values that quantify a state of an individual represented by the treatment profile. For example, each treatment profile can include indicators captured at a point in time at which an individual is suspected of or diagnosed as having the disease. Additionally, or alternatively, each treatment profile can include information about an age of the individual when the indicators were measured (e.g., through a laboratory system and/or a sequencing system that are the same as, or similar to, the laboratory system and sequencing system of FIG. 1).

In some embodiments, the analytics server can be configured to filter the first plurality of entries based on one or more criteria. For example, the analytics server can filter the first plurality of entries based on criteria indicating administration or non-administration of an agent or collection of agents (e.g., venetoclax (VEN) and azacitidine (AZA)) to the individuals represented by the first dataset. In this example, the analytics server can be configured to filter the first plurality of entries based on whether the individuals were treated using the agent or collection of agents to adjust progression of the disease. In some embodiments, the analytics server can be configured to filter the first plurality of entries based on one or more other criteria. For example, the analytics server can be configured to filter the first plurality of entries based on an age of the individuals represented by the first plurality of entries. In these examples, the analytics server can then determine a second plurality of entries in response to filtering the first plurality of entries.

At operation 404, the analytics server can determine a set of features from among a plurality of features. For example, the analytics server can determine a set of features corresponding to indicators represented by the treatment profiles of the first plurality of entries or the second plurality of entries. For example, the analytics server can determine a set of features that correspond to indicators generated in accordance with one or more techniques implemented by the laboratory system and/or the sequencing system. In this example, the analytics server can determine the set of features based on measurements and analysis of samples of the individuals obtained and processed by one or more of: flow cytometric systems, cytogenic systems, fluorescence in situ hybridization systems, next generation sequencing systems, and/or the like.

The analytics server can execute feature selection operations to identify a subset of variables that fall between a top threshold and a bottom threshold of association with progression indicators. In some embodiments, the analytics server can determine correlation coefficients or hazard-based association metrics for each variable and/or feature represented within treatment profiles accessible by the analytics server. In this example, the analytics server can determine a feature importance score for each variable and/or feature, such as a biomarker, clinical attribute, or genomic mutation, by quantifying the correlation between the variable and the likelihood of disease progression, resulting effects attributable to administration of a therapy, etc. Features with importance scores above the top threshold or below the bottom threshold can be excluded as being overly dominant or uninformative. In at least some examples, the analytics server can determine the thresholds using percentile-based ranking derived from statistical association tests and can select features whose scores lie between those boundaries to improve signal stability across model training iterations. For example, the analytics server can identify covariates exhibiting strong correlation with survival events at intermediate magnitudes, indicating balanced predictive contribution to model fitting. The resulting selected feature set can be used by the analytics server (e.g., through execution of a model development environment as described herein) to train (e.g., fit) one or more predictive models that estimate probabilities associated with disease progression or response to administered therapies.

At operation 406, the analytics server can generate an analysis dataset comprising a second plurality of entries based on the first dataset. For example, the analytics server can generate the analysis dataset based on the first dataset and the set of features identified by the analytics server as satisfying (e.g., being compatible with) a given model to be trained and/or fitted. In one example, where a model is being trained and/or fitted to predict health outcomes (e.g., survival rates at a point or points in time after the individual was treated or not treated with a therapy) based on indicators generated using a particular system and/or technique (e.g., a flow cytometric system), the analytics server can generate a portion of the analysis dataset to use when training and/or fitting that model. In this example, the analytics server can include the features represented in the first plurality of entries that correspond to indicators derived from the flow cytometric systems, cytogenic systems, fluorescence in situ hybridization systems, next generation sequencing systems, and/or the like.

In some embodiments, the analytics server can augment the analysis dataset to include one or more sub-entries corresponding to each entry. For example, the analytics server can augment the analysis dataset to generate a plurality of sub-entries for one or more of the second plurality of entries, where each sub-entry includes one or more synthetic indicators that augment the indicators for the respective one or more of the second plurality of entries. The analytics server can determine these synthetic indicators based on execution of one or more resampling techniques (e.g., such as category assignment, complete case inverse weighting, and/or multiple imputation by chained equations, etc.).

At operation 408, the analytics server can train and/or fit a plurality of models based on the analysis dataset. For example, the analytics server can train a plurality of models based on the entries and/or sub-entries included in the analysis dataset. In this example, the analytics server can determine combinations of entries and/or sub-entries in the analysis dataset that satisfy the configuration of a particular model and train and/or fit that particular model based on the corresponding portions of the analysis dataset. In some embodiments, the analytics server can execute each model based on different functional architectures corresponding to predetermined analytical objectives. For example, the analytics server can train a penalized regression model based on continuous covariates such as expression-level biomarkers and a tree-based ensemble model based on categorical or binary inputs representing discrete mutation states. In some examples, the analytics server can apply layer-wise updates during training iterations, where each update modifies optimization parameters such as learning rate, regularization strength, and loss convergence tolerance based on performance of a given model during training. In this example, the analytics server can iterate through cross-validation partitions of the analysis dataset to calculate intermediate parameter updates and store the resulting fitted coefficients or decision weights for subsequent performance comparison with other trained models. In at least some examples, the analytics server can assign a unique model identifier to each trained instance and cache corresponding prediction residuals, weighting matrices, or feature effect vectors for further ranking of model precision under different training configurations.

At operation 410, the analytics server can determine, for each individual represented by the subset of entries, a plurality of performance metrics. For example, the analytics server can determine, for each individual, performance metrics representing predictions of survival at a point in time after disease diagnosis and/or whether the individual experienced one or more side effects as a result of the administration or non-administration of a therapy. The analytics server can then determine an average performance metric for each individual. For example, where the analytics server trains and/or fits a plurality of treatment profiles of the analysis dataset to a plurality of models, the analytics server can then determine the metrics based for each individual using the plurality of models by selecting the mean value or averaging the values for a given metric output by the plurality of models. The analytics server can then determine a plurality of probabilities that the individual will survive to one or more points in time and average these probabilities to determine an average performance metric.

In some embodiments, the analytics server can determine a treatment response classification for each individual based on the performance metrics associated with that individual. For example, analytics server can determine an average performance metric corresponding to each individual to determine a treatment response classification. The analytics server can then determine an administration response for each individual based on the average performance metric corresponding to each individual. The treatment response can indicate whether the individual is predicted to have a strongly adverse treatment response, a moderately adverse treatment response, a neutral treatment response (signaling adversity), a neutral treatment response (signaling favorability), a moderately favorable treatment response, or a strongly favorable treatment response.

At operation 412, the analytics server can determine, for each individual represented by the subset of entries, an administration response. For example, the analytics server can determine an administration response for each individual, where the administration response indicates a favorable, intermediate, or not favorable response. In some embodiments, the analytics server can derive the administration response by comparing the predicted probability distributions of the trained models against predefined survival thresholds representing observed clinical outcomes within the analysis dataset. For example, the analytics server can calculate a probability score associated with the survival likelihood of each individual based on the model outputs and map the score to categorical intervals corresponding to favorable, intermediate, or not favorable classifications. In examples, probability scores equal to or greater than an upper threshold can correspond to favorable responses, scores between the upper and lower thresholds can correspond to intermediate responses, and scores lower than the lower threshold can correspond to not favorable responses. In at least some examples, the analytics server can execute a smoothing function, such as a weighted averaging algorithm, to refine continuous response probabilities into discrete categorical outcomes generated for each subject. In this example, discrete classifications can be produced for each therapy type evaluated by the analytics server, allowing the administration response to be expressed as a categorical label associated with each treatment profile included in the analysis dataset. The analytics server can then cause a user interface to be displayed to indicate whether to administer a therapy (such as ven/aza in the case of AML where an individual is not eligible for IC).

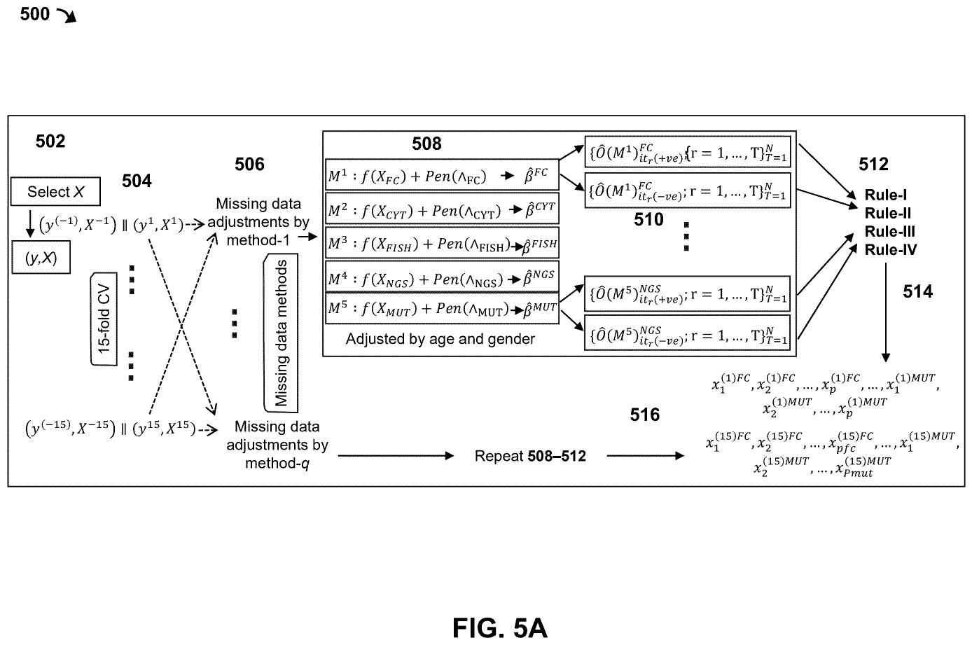

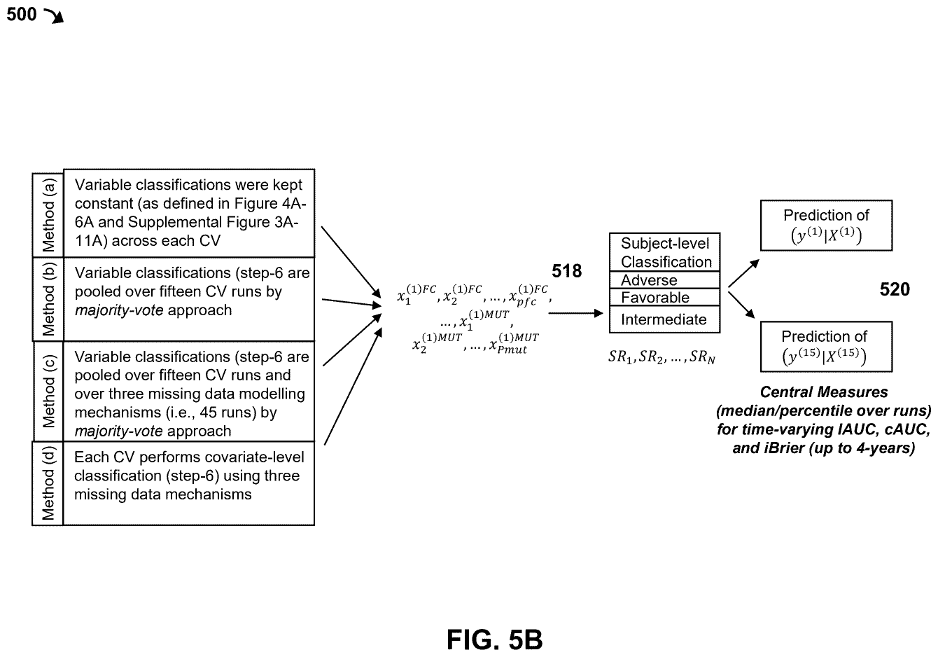

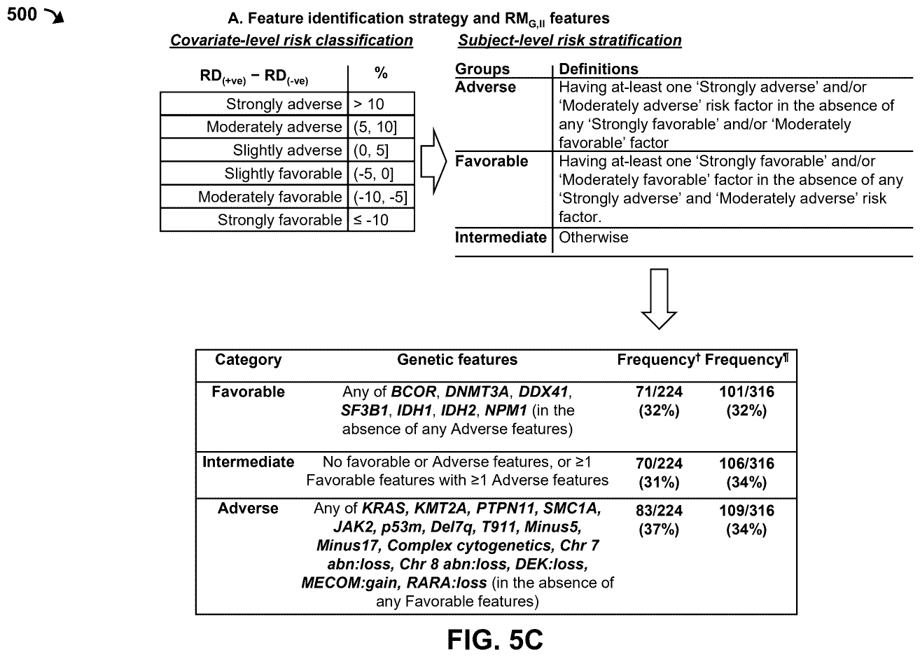

FIGS. 5A-5C are a diagram of an example implementation of a process 500 for configuring and validating predictive performance of models to determine whether administration of a therapy will or will not result in adverse or non-adverse outcomes, in accordance with one or more embodiments described herein. In some embodiments, the operations of the process 500 can be implemented by an analytics server that is the same as, or similar to, the analytics server 102 of FIG. 1. While one or more operations are described as being performed by one or more devices as described herein, it will be understood that any suitable device described herein can implement one or more of the operations described herein, alone or in coordination with one or more other devices described, unless context clearly indicates otherwise. In examples, one or more of the operations described with respect to process 500 can be the same as, or similar to, operations described with respect to the method 400 of FIG. 4.

In the context of acute myeloid leukemia (AML), this condition is an aggressive hematopoietic malignancy that is diagnosed in ˜20,000 people per year in the United States (US). For young and fit patients, treatment can involve aggressive intensive chemotherapy (IC) initially followed by consolidation with allogeneic hematopoietic stem cell transplant (HSCT) or additional high dose chemotherapy. For older and less fit patients, venetoclax (ven) combined with a hypomethylating agent (HMA) such as azacitidine (aza) or decitabine can be implemented. For IC therapies, a variety of prognostic models can be used based on patient and AML features to stratify patients into subgroups with varying outcomes.

While certain risk models (RMs) were developed, such as the European Leukemia Network (ELN) RM for AML (ELN17) (updated in 2022 (ELN22)), these RMs are configured to allow for determination of overall survival (OS) based on a set of features that, by virtue of their configuration, do not perform well for ven/HMA treated patients, likely because the ELN criteria are largely based on treatment outcomes following IC. Newer RMs can be implemented as described herein for lower intensity therapies including ven/HMA to both address deficiencies in model performance (noted above) while also capturing real-world data (RWD) challenges including missingness, sparsity, scarcity, type of end-points, collinearity, and the impact of hematopoietic stem cell transplant (HSCT) on OS. In addition, the described models are not limited by fixed rules and assumptions and are readily adaptable as technologies and findings evolve.

While the techniques described herein are discussed with respect to the development of a machine learning based AML risk stratification strategy for newly diagnosed AML patients treated specifically with ven/aza based on a wide range of AML diagnostic pathology features. In addition, this strategy addresses a variety of RWD challenges and is readily tunable and flexible. While certain aspects regarding these techniques are described with respect to AML, it will be understood that any other suitable condition can be analyzed to indicate one or more treatments based on the analysis of treatment profiles to generate risk stratifications for subsequent classification and treatment.

With continued reference to FIGS. 5A-5C, at operation 502, the analytics server can perform feature selection to identify one or more features based on (e.g., from) an available dataset (e.g., a dataset that is the same as, or similar to, the global patient database 110 and/or the refined datasets 108 of FIG. 1) representing individual-level observations (e.g., data aggregated or derived from individual patients representing measurable clinical, demographic, genomic, or treatment-related variables collected across observation periods). For example, the analytics server can obtain observed (e.g., measured, documented, etc.) values for indicators as described herein and determine candidate features usable to train models and correlated with survival outcomes or treatment response signals to one or more therapies. These outcomes can be associated with the administration of therapies such as venetoclax and azacitidine (ven/aza) to individuals having AML in comparison to individuals that are not treated with the one or more therapies. In some embodiments, the analytics server can perform this determination by executing univariate or multivariate filtering, bootstrapping, or ridge-penalized regression across individual-level features represented in the treatment profiles accessible by the analytics server to isolate indicators contributing variation in corresponding feature values or groups of feature values represented in the dataset across individuals treated as compared to those are untreated with a therapy. In this example, each regression iteration can result in the generation of coefficient vectors representing relationships between input covariates (e.g., measurable indicators such as variables, attributes, or features representing individual-specific characteristics from a treatment profile such as genomic, clinical, or demographic data used in modeling or statistical analysis) and response endpoints (e.g., overall survival, event-free survival, etc.), allowing the analytics server to establish feature importance rankings. In some embodiments, the analytics server can execute this feature selection prior to cross-validation partitioning described herein so that subsequent model training uses harmonized input fields that satisfy threshold inclusion criteria (e.g., predefined quantitative or statistical conditions used to determine whether a feature, variable, or data element is retained for analysis or model training). In examples, the analytics server can evaluate the statistical significance of candidate variables using null hypothesis significance testing and may apply Kaplan-Meier-derived log-rank calculations to confirm a set of indicators to be used in downstream analysis before model fitting.

At operation 504, the analytics server can adjust for missing data (e.g., in the treatment profiles of the datasets accessible by the analytics server). For example, the analytics server can estimate inverse probability weights (IPWs) using a generalized linear model (GLM) applied to a subset of complete treatment profiles (e.g., treatment profiles having durations that extend over a period of time, such as a period when a therapy was administered or not administered after detection of a condition such as AML) within the training dataset. In this operation, individuals with fully observed feature values (e.g., covariate values representing biomarkers observed for the individuals) can be assigned subject-specific weights by the analytics server based on probabilities derived from the output generated by the GLM. In some embodiments, the analytics server can apply a weighted modeling process to adjust for potential bias introduced by incomplete cases (e.g., treatment profiles that do not span the entire period of time after detection of the condition) that are excluded from the IPW estimation. In this example, the GLM can receive input vectors representing covariate patterns for each complete record and return predicted probabilities that are subsequently inverted to define respective IPWs for downstream processing.

At operation 506, the analytics server can update the one or more treatment profiles having missing data. For example, the analytics server can adjust for missing data following the initial estimations of inverse probability weights for complete cases. To do so, the analytics server can apply independent imputation and weighting adjustments, repeating the evaluation procedure across all variables within the treatment profiles. In examples, the analytics server can generate adjustments by combining outputs for specific variables (e.g., specific indicators or features) that are missing in certain treatment profiles through the implementation of one or more missing data strategies, such as category assignment, complete case inverse weighting, and/or multiple imputation by chained equations (MICE). For example, the analytics server can execute five sequential imputations for each incomplete covariate using preceding values within a feature space X (e.g., a multidimensional data structure representing all measurable variables, covariates, or features available for model training and analysis) under an impute-then-select approach to reconstruct missing indicators prior to final model fitting. In this example, processed versions of the same dataset, each subject to a different missing-data adjustment, can be treated as separate analytical variants indexed from 1-q, where q corresponds to the specific imputation or weighting approach executed. The analytics server can identify, across these variants, deviations in marginal risk estimates and aggregate the resulting values through an ensemble or majority-vote sequence, generating a harmonized dataset for downstream survival regression or counterfactual modeling. In some examples, each adjustment instance can preserve covariate-level consistency by maintaining patient identifiers and endpoint alignment across resampling iterations (e.g., by not adjusting these indicators).

At operation 508, the analytics server can execute penalized Cox proportional hazard regressions for each type of feature value represented in the treatment profiles (referred to as biomarker types or indicator types) independently. In some embodiments, the analytics server can perform separate model fitting tasks for indicators associated with next generation sequencing features, cytogenetic features, fluorescence in situ hybridization features, and mutation-derived covariates derived from the treatment profiles in the analysis dataset. In this example, the analytics server can control model sparsity using a ridge penalty term associated with a time-to-event response and tune hyperparameters through a cross-validation process that identifies regularization values minimizing prediction error across training iterations. In examples, the analytics server can adjust each regression for covariates such as age and gender (e.g., by filtering, etc.) and compute fractional bootstrap estimates through repeated re-sampling procedures to obtain stable confidence intervals around the hazard coefficients (e.g., numerical estimates representing the relative effect or contribution of individual covariates to the event rate associated with a given model). The analytics server can then output coefficient vectors calculated for each feature type (e.g., biomarker category), indicating relative prognostic weights and reliability intervals for those features across cross-validation runs. In at least some examples, the analytics server can implement these fitted regressions to produce counterfactual risk profiles used in subsequent marginal risk and classification operations.

At operation 510, the analytics server can determine recycled predictions representing mean marginal probabilities of risk over time using counterfactual conditions for each treatment profile. For example, the analytics server can calculate, for each feature type represented in a given treatment profile, predicted marginal probabilities under two hypothetical states, one assuming that a therapy (e.g., ven/aza) was administered and another assuming that the therapy was not administered. In some embodiments, each prediction can be generated using model coefficients derived from the penalized Cox proportional hazard regressions. For example, the analytics server can determine subject-level marginal probabilities for survival (e.g., overall survival, event-free survival, etc.) or event occurrence by substituting adjusted hazard ratio estimates corresponding to the administration or non-administration of the therapy into the fitted models (see operation 508). In this example, the analytics server can use a fixed patient cohort when averaging marginal probabilities to preserve sample consistency and account for sparsity in biomarker representation. In at least some examples, the analytics server can repeat this determination across multiple bootstrap samples (e.g., resampled subsets of treatment profiles generated with replacement from an original dataset to approximate variability and estimate confidence intervals), aggregating results over a number of resampling iterations (e.g., 2000 iterations, etc.) to obtain stable mean marginal risk profiles. The analytics server can then store the resulting time-varying marginal probabilities associated with the administration or non-administration of the therapy as counterfactual risk estimates that serve as inputs to subsequent risk difference and covariate-level classification operations.

At operation 512, the analytics server can obtain bias-corrected 95% confidence intervals (CIs) for each of the computed estimates, marginal risks, and risk differences based on the outputs generated from the prior survival regression and counterfactual estimation. The analytics server can calculate the bias-corrected 95% CIs using a percentile-based bootstrap distribution derived from fractional random weight bootstrap (FRWB) resampling executed over a predetermined number of iterations, such as 2,000 runs. In some embodiments, each bootstrap iteration can produce a distribution of parameter estimates associated with adjusted hazard ratios or marginal probabilities under the modeled treatment and counterfactual conditions. For example, the analytics server can identify the upper and lower percentile bounds corresponding to the 2.5th and 97.5th percentiles of the cumulative frequency distribution of each parameter estimate and can store those bounds as the bias-corrected confidence limits. In at least some examples, the confidence intervals (e.g., numerical ranges derived from repeated sampling or statistical estimation that indicate the degree of uncertainty or reliability of a determined parameter in predicting whether individuals associated with partial or complete treatment profiles will respond (and the degree to which they will respond) to a given therapy)) can be determined separately for each feature type, such as next generation sequencing, cytogenetics, fluorescence in situ hybridization, and mutation-based features, among others, allowing quantification of estimation uncertainty across feature-specific hazards. The analytics server can perform this operation after determining counterfactual mean marginal risk profiles so that subsequent risk difference calculations incorporate these bias-corrected statistical bounds.

At operation 514, the analytics server can determine covariate-level risk differences based on the counterfactual estimates. In some embodiments, the analytics server can calculate the risk difference for each variable by subtracting the marginal survival probability derived under a negative state (e.g., where a therapy is not administered) from the marginal survival probability derived under a positive state (e.g., where a therapy is administered). In this example, the analytics server can determine at a predefined temporal landmark (e.g., 3 months, 6 months, 14.7 months, etc.) corresponding to the median observation interval in the analysis dataset. The analytics server can aggregate results across bootstrap resampling iterations and generate mean risk difference values for each feature type. In some examples, the analytics server can compare each determined risk difference value against a predefined 5 percent differential threshold and apply categorical assignment rules to classify covariates as favorable, neutral, or adverse when indicating whether a treatment profile of an individual will or will not respond to the treatment. In this example, the analytics server can store the resulting classifications as covariate-level stratification outputs that are then used to determine subject-level classification (e.g., whether a given individual will have a favorable, neutral, unfavorable, etc., response to the therapy).

At operation 516, the analytics server can repeat operations 508, 510, and 512 when calculating updated risk differences associated with a temporal landmark (e.g., as new treatment profiles are obtained or otherwise included in the datasets accessible by the analytics server). In some embodiments, the analytics server can execute repeated survival regression and counterfactual estimation procedures over resampled patient subsets to refine bootstrap-based marginal risk estimates. In this example, the analytics server can recalculate marginal probabilities for each positive and negative response using ridge-penalized model parameters derived from the prior iteration. In examples, averaged risk difference values can be generated across a plurality of resampling runs until convergence criteria are met for the mean differential threshold. For example, the analytics server can iteratively generate counterfactual survival probabilities under hypothetical administration and non-administration of a therapy and update time-adjusted marginal profiles to reflect observed feature persistence through an observation window (e.g., of 3 months, 6 months, 14.7 months, etc.). In at least some examples, the analytics server can aggregate these temporally adjusted risk difference estimates into a new structured dataset accessible by the analytics server when performing downstream subject-level classification.

At operation 518, and with reference to FIG. 5B, the analytics server can apply logic to determine subject specific risk stratification across one or more versions of one or more fitted models. For example, the analytics server can process individual covariate-level classifications produced during each cross-validation run and aggregate them into a single individual-level output (e.g., score) for a given treatment profile analyzed by the analytics server. In doing so, the analytics server can merge the covariate-level scores associated with one or more feature types, such as features representing indicators such as next generation sequencing, cytogenetics, or flow cytometry into composite representations of treatment response for each subject. In this example, the analytics server can apply Boolean assignment logic that combines the sign and magnitude of feature-specific risk differences to classify treatment profiles for individuals into categorical outcomes such as favorable, intermediate, or adverse. While these three groups are discussed, it will be understood that any number of groups can be used. For example, the analytics server can be configured to classify treatment profiles as “Strongly adverse,” “Moderately adverse,” “Slightly adverse,” “Slightly favorable,” “Moderately favorable,” or “Strongly favorable.” In this example, scores can be grouped such that treatment profiles are scored along a continuum from 10 or more (adverse) to −10 or less (favorable). For example, treatment profiles indicating strongly adverse risk (e.g., with proportionally higher probabilities of patients experiencing risk factors (e.g., measurable clinical, demographic, genomic, or environmental variables associated with the likelihood of disease progression or treatment response after administration of a therapy)) can have scores of 10 or more, Moderately adverse can have scores between 5 and 10, Slightly adverse can have scores between 0 and 5, Slightly favorable can have scores between 0and −5, Moderately favorable can have scores between −5 and −10, and strongly favorable can have scores that are less than −10. As will be understood, these risk factors can represent the proportional risk to benefit when comparing administration of a therapy (such as ven/aza) to the non-administration of the therapy. It will be understood that adverse risk stratification can indicate that a given treatment profile is associated with one or more “adverse” risk factors in the absence of, or in combination with, one or more “favorable” or “intermediate” risk factors, or vice versa.

In some examples, the analytics server can assign each subject to a particular group based on the presence or absence of covariates that were previously labeled as favorable or adverse under one or more rules (e.g., Rule I, Rule II, Rule III, or Rule IV corresponding to sets of covariate-level classification criteria defining how marginal risk differences are computed and categorized when developing and validating risk stratification models). These rules can include Rule I (that variable classifications were kept constant across each cross-validation execution); Rule II (that variable classifications are pooled over a predetermined number of cross-validation executions using a “majority vote” approach; Rule III (that variable classifications are pooled over a predetermined number of cross-validation executions and over three missing data modeling mechanisms (e.g., 45 runs using a “majority vote” approach); and/or Rule IV (that each cross-validation execution involves performing covariate-level classifications using a predetermined number or group of missing data imputation techniques, for example, those described above). In at least some examples, the assignment decision can occur after all marginal risk differences and bootstrap-derived confidence intervals have been finalized, allowing for each individual entry in the analysis dataset to be assigned a single categorical value aligned with the corresponding distribution of model-derived probabilities.

Rule I can apply a point-estimate procedure to determine mean marginal risks for each feature at a specified temporal landmark. For example, the analytics server can calculate a mean marginal risk difference between positive and negative states associated with administration or non-administration of a therapy without exclusion of any variables irrespective of representation frequency thresholds. In some embodiments, point-estimates can be derived from a single model iteration using hazard coefficients and bootstrap-generated confidence limits associated with each feature type. In this example, every feature represented within the dataset can have equal eligibility for classification regardless of its prevalence within the analyzed cohort. Rule II can apply a prevalence-based exclusion to compute mean marginal risks while omitting variables that occur below or above specific representation frequencies. For example, the analytics server can disregard features that are present in cases within ranges less than a lower threshold or greater than an upper threshold of representation in the treatment profiles (e.g., where the features have positive values that are present in up to 2% of the treatment profiles accessible by the analytics server or in up to 40% of the treatment profiles). In this example, the exclusion criteria can prevent skewing of subject-level classifications by features that are either too sparse to yield reliable estimates or too common to discriminate treatment response differences. In some embodiments, remaining variables can be processed using the same point-estimation approach implemented in Rule I. Rule III can apply a bootstrap-based averaging strategy that repeats risk estimation across multiple resampling runs to stabilize the variance of marginal risk assessments. For example, the analytics server can generate two thousand independent bootstrap iterations of each feature set and compute the mean marginal risks by averaging profiles over all iterations. In some embodiments, this operation can produce fractional uncertainty intervals that more accurately represent individual-level estimation error associated with each coefficient. In this example, features can remain included regardless of prevalence but undergo smoothing across bootstrap realizations. Rule IV can apply a hybrid approach combining the prevalence constraints of Rule II with the resampling and averaging strategy of Rule III. For example, the analytics server can first remove variables falling outside the two to forty percent representation threshold and subsequently execute two thousand bootstrap iterations on the remaining subset of features. In this example, resulting mean marginal risk estimates can exhibit both reduced representation bias and improved estimation stability due to resampling aggregation. In some embodiments, the analytics server can use these hybrid-derived risk metrics to assign variable-level classifications that serve as input to downstream subject-level risk stratification.

The model architectures that can be implemented in the described system can include penalized regression models, tree-based machine learning models, and multilayer neural network models, among others. In some examples, the analytics server can execute penalized Cox proportional hazard models using ridge penalties to regulate coefficient magnitudes and prevent overfitting during time-to-event predictions. Tree-based models can include decision trees or gradient boosting frameworks trained to partition treatment profiles along the most informative features, producing interpretable decision boundaries that capture non-linear relationships between covariates and survival outcomes. Neural network models, on the other hand, can include fully connected feed-forward networks configured to learn complex, high-dimensional patterns from the treatment profiles. Each architecture can be independently trained and evaluated under the covariate-level classification rules such as Rule I through Rule IV to determine which configuration most accurately distinguishes the presence or absence of treatment-related risk factors in the analyzed group of treatment profiles accessible by the analytics server.

The models can be configured to receive, as inputs, treatment profiles representing patient-level observations drawn from biological and clinical data sources as described herein. Each treatment profile can include indicators such as genetic mutation states, cytogenetic alterations, fluorescence in situ hybridization findings, and phenotypic measurements obtained from flow cytometry. These indicators can be expressed numerically or categorically and can serve as covariates reflecting characteristics of the disease progression in an individual at baseline (e.g., before therapy) and after therapy administration. The outputs of the models can include probabilities or categorical classifications that indicate whether one or more risk factors are predicted to be present when treatments are administered. In AML-specific implementations, for example, the model can output an indication of a risk category such as “favorable,” “intermediate,” or “adverse,” which corresponds to survival likelihood or treatment response classification determined across counterfactual and observed data.

Each model can be trained using the treatment profiles that have been preprocessed under one or more of the covariate-level classification rules. The training can involve presenting batches of treatment profile data as input, computing predicted probabilities for each class of outcome, and comparing those predictions to known clinical outcomes representing measured treatment responses. During training, the models can calculate gradients of loss functions representing the difference between predicted and actual outcomes, and the parameters or weights within the model can be updated by backpropagation or other suitable techniques to reduce error across iterations. This process can continue until convergence criteria are met, such as stabilization of loss or improvement of prediction accuracy satisfy a threshold value. The result is a trained model that, when applied to new grouping of treatment profiles, generates outputs indicating whether adverse, intermediate, or favorable risk factors are present under an administered therapy in accordance with the selected classification rule set.

At operation 520, the analytics server can evaluate predictive performance of one or more fitted models across a predetermined number of cross-validation runs using different covariate-level classification approaches. In some embodiments, the analytics server can apply statistical scoring routines to quantify the predictive stability and accuracy of the trained models across the fifteen folds executed in the model development environment. In examples, the analytics server can compute central performance measures such as time-dependent incident area under the curve (iAUC), cumulative area under the curve (cAUC), and integrated Brier scores (iBrier) representing temporal forecast reliability at predefined follow-up intervals. In this example, each cross-validation iteration can generate a distinct set of survival probabilities that the analytics server can aggregate into median or percentile distributions to reduce variance between runs. In at least some examples, the analytics server can execute ensemble averaging of the summarized metrics across multiple missing data adjustment variants to produce harmonized performance indicators. The analytics server can then establish final performance tables and comparison indices summarizing model precision and calibration consistency across overall survival and event-free survival endpoints derived from the analyzed treatment profiles.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of this disclosure or the claims.

Embodiments implemented in computer software can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents. Information, arguments, parameters, data, etc., can be passed, forwarded, or transmitted via any suitable means, including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate the transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes and/or instructions on a non-transitory processor-readable medium and/or computer-readable medium, which can be incorporated into a computer program product.

Some embodiments of the present disclosure are described herein in connection with a threshold. As described herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, and/or the like.

The preceding description of the disclosed embodiments is provided to enable any person skilled in the art to make or use the embodiments described herein and variations thereof. Various modifications to these embodiments will be readily apparent to those skilled in the art, and the principles defined herein can be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A system for selecting an optimized system configuration based on performance metrics associated with time-to-event determinations generated by a plurality of systems using treatment profiles, the system comprising:
one or more processors configured to:
determine a set of features from among a plurality of features represented across a plurality of treatment profiles, the set of features correlated with time-to-event determinations that satisfy a correlation threshold;
generate an analysis dataset comprising a plurality of second treatment profiles based on the set of features that are correlated with the time-to-event determinations;
train a plurality of models based on the analysis dataset to output the time-to-event determinations;
determine, for each model of the plurality of models configured with the analysis dataset or a different dataset, a plurality of performance metrics based on the time-to-event determinations;
configure the system to receive different treatment profiles to determine different time-to-event determinations by:
selecting an optimized model from among the plurality of models based on the plurality of performance metrics; and
for at least one treatment profile, generating an indication of a therapy response based on the optimized model and a time-to-event determination generated by the optimized model, the time-to-event determination representing a counterfactual analysis of the treatment profile in a first case where a therapy is administered and in a second case where a therapy is not administered.

2. The system of claim 1, wherein the set of features represent individuals having a condition,
wherein the one or more processors are further configured to:
filter the plurality of features based on one or more criteria indicating administration of an agent that is configured to adjust progression of the condition; and
determine the set of features in response to filtering the plurality of features.

3. The system of claim 2, wherein the one or more processors configured to filter the plurality of features are configured to:
filter the plurality of features based on a plurality of criteria indicating administration of a plurality of agents that are configured to adjust progression of the condition when administered in combination.

4. The system of claim 1, wherein the one or more processors configured to generate the analysis dataset are configured to:
generate a plurality of additional features for one or more of the plurality of second treatment profiles, each additional feature comprising one or more synthetic features that augment the features for the respective one or more of the plurality of second treatment profiles.

5. The system of claim 4, wherein the one or more processors configured to train the plurality of models are configure to:
for each model:
determine a combination of features and additional features that satisfy each model of the plurality of models; and
train the plurality of models using the combination of features and additional features.

6. The system of claim 1, wherein each performance metric of the plurality of performance metrics comprises a prediction of survival at a point in time after diagnosis of a condition,
wherein the one or more processors configured to determine the plurality of performance metrics for each profile are configured to:
determine an average performance metric corresponding to each profile to determine a treatment response classification,
wherein the one or more processors configured to determine the indication of the therapy response are configured to:
determine, for each profile, an administration response based on the average performance metric corresponding to each profile.

7. The system of claim 6, wherein the one or more processors configured to determine the administration response for each profile are configured to:
classify the administration response for each profile as being favorable, intermediate, or not favorable based on the average performance metric for each profile.

8. A method for selecting an optimized system configuration based on performance metrics associated with time-to-event determinations generated by a plurality of systems using treatment profiles, the method comprising:
determining, by one or more processors, a set of features from among a plurality of features represented across a plurality of treatment profiles, the set of features correlated with time-to-event determinations that satisfy a correlation threshold;
generating, by the one or more processors, an analysis dataset comprising a plurality of second treatment profiles based on the set of features that are correlated with the time-to-event determinations;
training, by the one or more processors, a plurality of models based on the analysis dataset to output the time-to-event determinations;
determining, by the one or more processors, for each model of the plurality of models configured with the analysis dataset or a different dataset, a plurality of performance metrics based on the time-to-event determinations;
configuring, by the one or more processors, the system to receive different treatment profiles to determine different time-to-event determinations by:
selecting, by the one or more processors, an optimized model from among the plurality of models based on the plurality of performance metrics; and
for at least one treatment profile, generating, by the one or more processors, an indication of a therapy response based on the optimized model and a time-to-event determination generated by the optimized model.

9. The method of claim 8, wherein the set of features represent individuals having a condition,
the method further comprising:
filtering, by the one or more processors, the plurality of features based on one or more criteria indicating administration of an agent that is configured to adjust progression of the condition; and
determining, by the one or more processors, the set of features in response to filtering the plurality of features.

10. The method of claim 9, wherein filtering the plurality of features comprises:
filtering, by the one or more processors, the plurality of features based on a plurality of criteria indicating administration of a plurality of agents that are configured to adjust progression of the condition when administered in combination.

11. The method of claim 8, wherein generating the analysis dataset comprises:
generating, by the one or more processors, a plurality of additional features for one or more of the plurality of second treatment profiles, each additional feature comprising one or more synthetic features that augment the features for the respective one or more of the plurality of second treatment profiles.

12. The method of claim 11, wherein training the plurality of models comprises:
for each model:
determining, by the one or more processors, a combination of features and additional features that satisfy each model of the plurality of models; and
training, by the one or more processors, the plurality of models using the combination of features and additional features.

13. The method of claim 8, wherein each performance metric of the plurality of performance metrics comprises a prediction of survival at a point in time after diagnosis of a condition,
wherein determining the plurality of performance metrics for each profile comprises:
determining, by the one or more processors, an average performance metric corresponding to each profile to determine a treatment response classification,
wherein determining the indication of the therapy response comprises:
determining, by the one or more processors, for each profile, an administration response based on the average performance metric corresponding to each profile.

14. The method of claim 13, wherein determining the administration response for each profile comprises:
classifying, by the one or more processors, the administration response for each profile as being favorable, intermediate, or not favorable based on the average performance metric for each profile.

15. One or more non-transitory computer-readable mediums storing instructions thereon that, when executed by one or more processors, cause the one or more processors to perform operations comprising:
determining a set of features from among a plurality of features represented across a plurality of treatment profiles, the set of features correlated with time-to-event determinations that satisfy a correlation threshold;
generating an analysis dataset comprising a plurality of second treatment profiles based on the set of features that are correlated with the time-to-event determinations;
training a plurality of models based on the analysis dataset to output the time-to-event determinations;
determining for each model of the plurality of models configured with the analysis dataset or a different dataset, a plurality of performance metrics based on the time-to-event determinations;
configuring a system to receive different treatment profiles to determine different time-to-event determinations by:
selecting an optimized model from among the plurality of models based on the plurality of performance metrics; and
for at least one treatment profile, generating an indication of a therapy response based on the optimized model and a time-to-event determination generated by the optimized model.

16. The one or more non-transitory computer-readable mediums of claim 15, wherein the set of features represent individuals having a condition,
wherein the instructions further cause the one or more processors to:
filter the plurality of features based on one or more criteria indicating administration of an agent that is configured to adjust progression of the condition; and
determine the set of features in response to filtering the plurality of features.

17. The one or more non-transitory computer-readable mediums of claim 16, wherein the instructions that cause the one or more processors to filter the plurality of features cause the one or more processors to:
filter the plurality of features based on a plurality of criteria indicating administration of a plurality of agents that are configured to adjust progression of the condition when administered in combination.

18. The one or more non-transitory computer-readable mediums of claim 15, wherein the instructions that cause the one or more processors to generate the analysis dataset cause the one or more processors to:
generate a plurality of additional features for one or more of the plurality of second treatment profiles, each additional feature comprising one or more synthetic features that augment the features for the respective one or more of the plurality of second treatment profiles.

19. The one or more non-transitory computer-readable mediums of claim 18, wherein the instructions that cause the one or more processors to train the plurality of models cause the one or more processors to:
for each model:
determine a combination of features and additional features that satisfy each model of the plurality of models; and
train the plurality of models using the combination of features and additional features.

20. The one or more non-transitory computer-readable mediums of claim 15, wherein each performance metric of the plurality of performance metrics comprises a prediction of survival at a point in time after diagnosis of a condition,
wherein the instructions that cause the one or more processors to determine the plurality of performance metrics for each profile cause the one or more processors to:
determine an average performance metric corresponding to each profile to determine a treatment response classification,
wherein the instructions that cause the one or more processors to determine the indication of the therapy response cause the one or more processors to:
determine, for each profile, an administration response based on the average performance metric corresponding to each profile.
